SEQUENCE LISTING (47 sequences)

SEQ ID NO: 1 - protein, 51 aa, Artificial Sequence
  NEDD9 AA 75-125
EQPASGLMQQTFGQQKLYQVPNPQAAPRDTIYQVPPSYQNQGIYQVPTGHG

SEQ ID NO: 2 - protein, 51 aa, Artificial Sequence
  NEDD9 AA 175-225
DVYDIPPSHTTQGVYDIPPSSAKGPVFSVPVGEIKPQGVYDIPPTKGVYAI

SEQ ID NO: 3 - protein, 12 aa, Artificial Sequence
  substrate domain P1
LYQVPNPQAAPR

SEQ ID NO: 4 - protein, 23 aa, Artificial Sequence
  NEDD9 substrate domain P2
GPVFSVPVGEIKPQGVYDIPPTK

SEQ ID NO: 5 - protein, 14 aa, Artificial Sequence
  peptide sequence
LYQVPNPQAAPRDT

SEQ ID NO: 6 - protein, 20 aa, Artificial Sequence
  peptide sequence
CFGQQKLYQVPNPQAAPRDT

SEQ ID NO: 7 - protein, 17 aa, Artificial Sequence
  peptide sequence
GEIKPQGVYDIPPTKGV

SEQ ID NO: 8 - protein, 18 aa, Artificial Sequence
  peptide sequence
CGEIKPQGVYDIPPTKGV

SEQ ID NO: 9 - protein, 14 aa, Artificial Sequence
  peptide sequence
KLYQVPNPQAAPRD

SEQ ID NO: 10 - protein, 25 aa, Artificial Sequence
  peptide sequence
KGPVFSVPVGEIKPQGVYDIPPTKG

SEQ ID NO: 11 - protein, 6 aa, Artificial Sequence
  peptide sequence
CFGQQK

SEQ ID NO: 12 - DNA, 21 bp, Artificial Sequence
  NEDD9 siRNA Sense 1
ggauccuacaccagucuaatt

SEQ ID NO: 13 - DNA, 21 bp, Artificial Sequence
  NEDD9 siRNA Antisense 1
uuagacugguguaggaucctt

SEQ ID NO: 14 - DNA, 21 bp, Artificial Sequence
  NEDD9 siRNA Sense 2
gaaggacaugugaucucaatt

SEQ ID NO: 15 - DNA, 21 bp, Artificial Sequence
  NEDD9 siRNA Antisense 2
uugagaucacauguccuuctt

SEQ ID NO: 16 - DNA, 21 bp, Artificial Sequence
  NEDD9 siRNA Sense 3
gcuauuggagagcaauucatt

SEQ ID NO: 17 - DNA, 21 bp, Artificial Sequence
  NEDD9 siRNA Antisense 3
ugaauugcucuccaauagctt

SEQ ID NO: 18 - RNA, 18 nt, Artificial Sequence
  HIF-1alpha siRNA 1
gaacaaauacauggguua

SEQ ID NO: 19 - RNA, 19 nt, Artificial Sequence
  HIF-1alpha siRNA 2
agaaugaaguguacccuaa

SEQ ID NO: 20 - RNA, 18 nt, Artificial Sequence
  HIF-1alpha siRNA 3
gauggaacacuagacaaa

SEQ ID NO: 21 - RNA, 19 nt, Artificial Sequence
  HIF-1alpha siRNA 4
caaguagccucuuugacaa

SEQ ID NO: 22 - DNA, 31 bp, Artificial Sequence
  Southern primer NEDD9 forward
tccacggtcgccaaggcattgtcccagggaa

SEQ ID NO: 23 - DNA, 26 bp, Artificial Sequence
  Southern primer WT reverse
gccatttagtatgtttgctttggggc

SEQ ID NO: 24 - DNA, 30 bp, Artificial Sequence
  Southern primer NEDD9-/- reverse
cggacttgaagaagtcgtgctgcttcatgt

SEQ ID NO: 25 - protein, 8 aa, Artificial Sequence
  partial peptide sequence
GPVFSVPV

SEQ ID NO: 26 - protein, 12 aa, Artificial Sequence
  partial peptide sequence
ESSLSASPAQDK

SEQ ID NO: 27 - protein, 23 aa, Artificial Sequence
  partial peptide sequence
DVYDIPPSHTTQGVYDIPPSSAK

SEQ ID NO: 28 - protein, 15 aa, Artificial Sequence
  partial peptide sequence
DGVYDVPLHNPPDAK

SEQ ID NO: 29 - protein, 13 aa, Artificial Sequence
  partial peptide sequence
SIGGTSGPHVGKK

SEQ ID NO: 30 - protein, 11 aa, Artificial Sequence
  partial peptide sequence
LFLDPDTAIER

SEQ ID NO: 31 - protein, 6 aa, Artificial Sequence
  partial peptide sequence
NAQLFK

SEQ ID NO: 32 - protein, 8 aa, Artificial Sequence
  partial peptide sequence
QVTAQDIR

SEQ ID NO: 33 - protein, 8 aa, Artificial Sequence
  partial peptide sequence
DLVDGINR

SEQ ID NO: 34 - protein, 10 aa, Artificial Sequence
  partial peptide sequence
LVFIGDTLTR

SEQ ID NO: 35 - protein, 7 aa, Artificial Sequence
  partial NEDD9 peptide sequence
EYLHFVK

SEQ ID NO: 36 - protein, 11 aa, Artificial Sequence
  partial NEDD9 peptide sequence
TAVDKVELFLK

SEQ ID NO: 37 - protein, 11 aa, Artificial Sequence
  partial NEDD9 peptide sequence
LFLDPDTAIER

SEQ ID NO: 38 - protein, 14 aa, Artificial Sequence
  partial NEDD9 peptide sequence
GVQFLEPPAETSEK

SEQ ID NO: 39 - protein, 38 aa, Artificial Sequence
  partial NEDD9 peptide sequence
DTIYQVPPSYQNQGIYQVPTGHGTQEQEVYQVPPSVQR

SEQ ID NO: 40 - protein, 10 aa, Artificial Sequence
  partial NEDD9 peptide sequence
LSFSSTGSTR

SEQ ID NO: 41 - protein, 29 aa, Artificial Sequence
  partial NEDD9 peptide sequence
RHQSLSPNHPPPQLGQSVGSQNDAYDVPR

SEQ ID NO: 42 - protein, 6 aa, Artificial Sequence
  RHQSLSPNHPPPQLGQSVGSQNDAYDVPR
HINEIR

SEQ ID NO: 43 - protein, 13 aa, Artificial Sequence
  partial NEDD9 peptide sequence
ESSLSASPAQSDK

SEQ ID NO: 44 - protein, 11 aa, Artificial Sequence
  partial NEDD9 peptide sequence
LVLDPDTAIER

SEQ ID NO: 45 - protein, 20 aa, Artificial Sequence
  partial NEDD9 peptide sequence
TFGQQKLYQVPNSQAASRDT

SEQ ID NO: 46 - protein, 18 aa, Artificial Sequence
  partial NEDD9 peptide sequence
CGEIKPQGVYDIPPTKGV

SEQ ID NO: 47 - protein, 18 aa, Artificial Sequence
  partial NEDD9 peptide sequence
VGEIKPQGVYDIPPTQGV

(19) United States
(12) Patent Application Publication
(10) Pub. No.: US 2021/0324058 A1
(43) Pub. Date: Oct. 21, 2021

(54) NEDD9 IN PULMONARY VASCULAR THROMBOEMBOLIC DISEASE

(71) Applicant: The Brigham and Women`s Hospital, Inc., Boston, MA (US)

(72) Inventors: George A. Alba, Boston, MA (US); Joseph Loscalzo, Dover, MA (US); Bradley A. Maron, Sharon, MA (US)

(21) Appl. No.: 17/290,960

(22) PCT Filed: Nov. 5, 2019
(86) PCT No.: PCT/US2019/059890

§ 371 Date: May 3, 2021

Related U.S. Application Data
(60) Provisional application No. 62/755,647, filed on Nov. 5, 2018.
(60) Provisional application No. 62/882,226, filed on Aug. 2, 2019.

(51) Int. Cl. C07K 16/18

(52) U.S. Cl. CPC C07K 16/18; A61K 2039/505

(57) ABSTRACT

Anti-NEDD9 antibodies and methods of making and using said antibodies.

### CLAIM OF PRIORITY

This application claims the benefit of U.S. Provisional Patent Application Ser. No. 62/755,647, filed on Nov. 5, 2018 and U.S. Provisional Patent Application Ser. No. 62/882,226, filed Aug. 2, 2019. The entire contents of the foregoing are hereby incorporated by reference.

### FEDERALLY SPONSORED RESEARCH OR DEVELOPMENT

This invention was made with Government support under Grant Nos. HL139019, HL131787, and HL139613 awarded by the National Institutes of Health. The Government has certain rights in the invention.

### TECHNICAL FIELD

Described herein are Anti-NEDD9 antibodies and methods of making and using said antibodies.

### BACKGROUND

Pulmonary vascular thromboembolism (PVTE) is a defining event underlying numerous clinically important diseases including: luminal pulmonary embolism (PE), cancer-associated PE, pulmonary arterial hypertension (PAH), and chronic thromboembolic pulmonary hypertension (CTEPH). It is estimated that PVTE affects >1,000,000 people in the United States annually and accounts for 1 in 8 deaths worldwide, corresponding to >$12 billion (USD) in healthcare costs annually (Silverstein et al. Arch Intern Med. 1998; 158:585-593). The mortality rate attributed to PVTE in unselected populations remains high at ˜30%, and in-hospital and 30-day mortality in tightly controlled clinical trials is ˜4% an 8%, respectively (Klock et al. Am J Respir Crit Care Med. 2010; 181:501-506). The current standard of care treatment for most PVTE is anticoagulant or thrombolytic drugs; however, these therapies do not target PVTE-specific molecular mechanisms. Moreover, these therapies are associated with an unacceptable rate of a major adverse clinical events due to off-target drug complications. From well-designed clinical trials with strict inclusion/exclusion criteria and careful adherence to guideline-based use of anticoagulants, 2-3% of thrombolysis patients are reported to have intracranial hemorrhage (ICH) that results in iatrogenic mortality in many cases. However, in “real world” practice, where at-risk patients are generally not screened out and adherence to guidelines is less strict, the prevalence of these events approaches 9.2% (Kasper et al. J Am Coll Cardiol 1997; 30:1165-71). Furthermore, the rate of major bleeding events inclusive of ICH and extracranial bleeding is much higher, with events occurring in 11.5% of thrombolysis patients in one recent clinical trial (Meyer et al. N Engl J Med 2014; 370:1402-11).

Increased platelet-endothelial adhesion is a key pathogenetic mechanism underlying chronic thromboembolic pulmonary hypertension (CTEPH), which is a highly morbid cardiovascular disease characterized by non-resolving pulmonary emboli, hypoxic vascular injury, and endothelial dysfunction.

### SUMMARY

Described herein are antibodies that bind specifically to human neural precursor cell expressed, developmentally down-regulated 9 (NEDD9) at an epitope in or near a NEDD9 substrate domain, e.g., a tyrosine rich substrate domain that is accessible on the extracellular HPAEC plasma membrane, e.g., a substrate domain that comprises one or more YxxP motifs, e.g., within one of the following sequences: NEDD9 AA 75-125: EQPASG LMQQTFGQQK LYQVPNPQAA PRDTIYQVPP SYQNQGIYQV PTGHG (SEQ ID NO: 1); or NEDD9 AA 175-225: DVYDIP PSHTTQGVYD IPPSSAKGPV FSVPVGEIKP QGVYDIPPTK GVYAI (SEQ ID NO:2), e.g., at an epitope in NEDD9 substrate domain P1, e.g., within LYQVPNPQAAPR (SEQ ID NO:3), or substrate domain P2, e.g., within GPVFSVPVGEIKPQGVYDIPPTK (SEQ ID NO:4). In some embodiments, the antibodies are (or are derived from) monospecific polyclonal antibodies or monoclonal antibodies.

Also provided herein are methods of generating an antibody that binds to an epitope in NEDD9 substrate domain. The methods comprise immunizing a mammal with a peptide comprising a sequence that is at least 80% identical to at least 10 consecutive amino acids from: (i) the NEDD9 substrate domain P1, e.g., a peptide comprising LYQVPNPQAAPR (SEQ ID NO:3), LYQVPNPQAAPRDT-amide (SEQ ID NO:5), or CFGQQKLYQVPNPQAAPRDT-amide (SEQ ID NO:6), or (ii) NEDD9 substrate domain P2, e.g., a peptide comprising GEIKPQGVYDIPPTKGV (SEQ ID NO:7) or CGEIKPQGVYDIPPTKGV-amide (SEQ ID NO:8), optionally wherein the peptide is modified to increase antigenicity, and collecting antibodies from the mammal. In some embodiments, the peptide is modified to increase stability or antigenicity, preferably wherein the peptide is conjugated to one or both of keyhole limpet hemocyanin or ovalbumin.

In some embodiments, the methods further include isolating the blood serum from the immunized mammal containing antibodies; isolating antibody-producing cells taken from the spleen or lymph node of the immunized mammal; fusing the isolated antibody-producing cells with myeloma cells resulting in a hybridoma; cloning the hybridoma and recovering antibody from the culture thereof to yield a monoclonal antibody; and purifying the monoclonal antibodies using NEDD9 or a peptide therefrom.

Also provided herein is an antibody that binds specifically to NEDD9, generated by a method described herein.

Further provided herein are antibodies that bind specifically to NEDD9, obtained from a mammal that has been immunized with a peptide comprising NEDD9 substrate domain P1 (LYQVPNPQAAPR) (SEQ ID NO:3) or NEDD9 substrate domain P2 (GPVFSVPVGEIKPQGVYDIPPTK; SEQ ID NO:4).

In some embodiments, the antibody reduces or blocks formation of binding complexes between NEDD9 and p-Selectin; reduces binding affinity of a protein-protein complex between NEDD9 and P-Selectin; and/or reduce PVTE formation and/or platelet-endothelial adhesion.

Additionally, provided herein are methods for reducing platelet-endothelial adhesion in a subject in need thereof, the method comprising administering to the subject a therapeutically effective amount of an antibody as described herein, e.g., made using a method described herein.

Also provided herein are methods of treating, or reducing risk of, pulmonary vascular thromboembolism (PVTE) in a subject in need thereof. The methods include administering to the subject a therapeutically effective amount of an antibody as described herein, e.g., made using a method described herein.

In some embodiments, the subject has, or is at risk of developing, luminal pulmonary embolism (PE), cancer-associated PE, pulmonary arterial hypertension (PAH), or chronic thromboembolic pulmonary hypertension (CTEPH).

In some embodiments, the methods include treating the subject with one or more of anticoagulation (warfarin, direct oral anticoagulants), systemic thrombolysis, catheter-directed thrombolysis, or surgical clot resection.

In some embodiments, the antibody is administered parenterally or orally.

Additionally, provided herein are the antibodies described herein for use in a method of treating, or reducing risk of, pulmonary vascular thromboembolism (PVTE) in a subject in need thereof, and for use in a method of reducing platelet-endothelial adhesion in a subject in need thereof.

In some embodiments, the subject has, or is at risk of developing, luminal pulmonary embolism (PE), cancer-associated PE, pulmonary arterial hypertension (PAH), or chronic thromboembolic pulmonary hypertension (CTEPH).

In some embodiments, the subject is also treated with one or more of anticoagulation (warfarin, direct oral anticoagulants), systemic thrombolysis, catheter-directed thrombolysis, or surgical clot resection.

In some embodiments, the antibody is formulated to be administered parenterally or orally.

Also provided herein are pharmaceutical compositions that include the antibodies as described herein.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-D. Hypoxia modulates HIF-1α-dependent upregulation of NEDD9 selectively in human pulmonary artery endothelial cells. (A) Anti-NEDD9 immunoblot and (B) immunofluorescence using NEDD9 Ab #1 was performed on lysates from human pulmonary artery endothelial cells (HPAECs) treated with normoxia or hypoxia (10%, 2%, and 0.2% O₂) for 24 hr (N=3) and quantified. IgG₁ was negative control. (C) Treatment with maximal hypoxia (0.2% O₂) for 24 hr did not affect NEDD9 expression significantly in human coronary artery endothelial cells (HCAECs) or human pulmonary artery smooth muscle cells (HPASMCs). In human brain microvascular endothelial cells (HBMVECs), hypoxia decreased NEDD9 expression compared to normoxia (N=3 for all experiments). (D) Compared to HPAECs transfected with vehicle control (Lipofectamine alone) (Lipo) or scrambled si-RNA (negative) control (si-Scr), transfection with si-HIF-1α for 24 hr decreased NEDD9 and inhibited hypoxia-induced upregulation of NEDD9 (N=3). UN, untreated; HIF, hypoxia-inducible factor. Representative immunoblots and micrographs are shown. a.u., arbitrary units. Data are presented as mean±SE. Scale bar=20 μm.

FIGS. 2A-F. The NEDD9 substrate domain is expressed on the extracellular plasma membrane of human pulmonary endothelial cells. (A) NEDD9 is a scaffolding protein and in Homo sapiens is comprised of 834 amino acids organized in four distinct domains: SH3, substrate domain, 411B, and C-terminal. Two NEDD9 cleavage peptide fragments (p55 and p65) have been reported previously.³⁷ To determine if either cleavage products corresponded to differences in NEDD9 localization in HPAECs, anti-NEDD9 immunofluorescence was performed using NEDD9 Ab #1 targeting the p55 fragment, and NEDD9 Ab #2 targeting the p65 fragment. (B) Compared to NEDD9 Ab #2, NEDD9 expression detected using NEDD9 Ab #1 was localized predominantly to the perimeter of cells (N=3). (C) The MS1 spectra from five abundant peptides (SEQ ID NOs: 26, 3, and 27-29, respectively) detected in trypsin-digested HPAECs lysates immunoprecipitated using NEDD9 Ab #1 corresponded exclusively to the p55 fragment, whereas (D) NEDD9 Ab #2 identified NEDD9 peptides (SEQ ID NOs: 30-34, respectively) corresponding to the p65 fragment (N=3). Red underline denotes a YxxP sequence. MS, mass spectrometry. (E) Compared to normoxia, hypoxia (0.2% O₂) for 24 hr increased co-localization of NEDD9 with the endothelial plasma membrane protein PECAM-1 analyzed using double immunofluorescence (N=3). (F) In HPAECs, NEDD9 expression was analyzed by immunoblot in plasma membrane fractions, confirmed by detection of Na⁺/K⁺ ATPase in the absence of (cytosolic) calreticulin (N=3). Calciretic; calcireticulin. Representative immunoblots and micrographs are presented. a.u., arbitrary units. Data are presented as mean±SE. Scale bar=40 μm.

FIGS. 3A-E. NEDD9 modulates platelet-endothelial adhesion without affecting platelet-platelet aggregation. (A) Platelets from healthy donor controls were analyzed by flow cytometry to confirm activation of platelets by thrombin receptor-activated peptide (TRAP) (10 μM) prior to HPAEC-platelet endothelial adhesion assays (N=3). (B) Compared to untransfected human pulmonary artery endothelial cells (HPAECs), si-NEDD9 decreased platelet-HPAEC adhesion under basal conditions and following TRAP stimulation of platelets. (C) Compared to wild type (WT) controls, the tail bleeding time in transgenic NEDD9^{-/-} mice was increased significantly under conditions of normoxia and following treatment of mice with hypoxia (10% O₂ for 5 d). (D) Anti-NEDD9 immunofluorescence (IF) and electron microscopy (EM) immunocytochemistry using NEDD9 Ab #3 were performed on platelets isolated from healthy human controls. For IF: scale bar=1.5 μm, and the scale bar for the magnified inset=10 μm. For EM: black arrows identify NEDD9 stain positivity (N=3). Scale bar=500 nm. (E) No significant difference between WT and NEDD9^{-/-} mice was observed for global platelet aggregation in response to collagen, protease activator receptor 4, or 9,11-Dideoxy-9α,11α-methanoepoxy prostaglandin F₂ (U46619) (N=3). N9, NEDD9. Representative micrographs and gating graphs are shown. Data are presented as mean±SE.

FIGS. 4A-E. P-Selectin binds the NEDD9 substrate domain. (A) HPAEC plasma membrane fractions were incubated with recombinant P-Selectin (0.5-1.0 μg), and liquid chromatography-mass spectrometry (LC-MS) was performed on samples following anti-P-Selectin immunoprecipitation. The MS2 spectra for each of the two detected NEDD9 peptide sequences, both within the substrate domain, are shown: K.LYQVPNPQAAPR.D (AA: 91-102; SEQ ID NO:9) (m z 677.36735 at retention time 28.1 s) (N9-P1) and K.GPVFSVPVGEIKPQGVYDIPPTK.G (AA: 191-211; SEQ ID NO:10) (m/z 808.77731 at retention time 35.5 s) (N9-P2) (N=2 replicates for N=2 iterations). Underlining indicates a YxxP sequence. (B) HPAEC plasma membrane fractions were incubated with exogenous P-Selectin and co-immunoprecipitation for P-Selectin and NEDD9 was performed. PM, plasma membrane; UN, untreated. Varying concentrations of P-Selectin (ligand) (2 μM-0.5 nM) were co-incubated with NEDD9 (receptor) (20 nM) and microscale thermophoresis was performed to assess macromolecular interactions between these proteins. (C) Raw fluorescence tracings, (D) capillary scan, and (E) dose titration curve show a high-quality analysis indicating definitive protein-protein interaction between the receptor and ligand (K_{D}=13.9±11.3 nM) (N=2). Representative immunoblot and titration curve are shown. Data are presented as mean±SE.

FIGS. 5A-E. NEDD9 inhibition with a monospecific anti-NEDD9 antibody prevents platelet-endothelial adhesion in vitro and pulmonary arterial thrombosis and pulmonary hypertension in vivo. (A) Immunofluorescence was performed on lung sections from WT C57BL/6 and NEDD9^{-/-} mice treated with normoxia (21% O₂) and hypoxia (10% O₂ for 5 d) using NEDD9 Ab #1, msAb-N9-P1, or msAb-N9-P2 (N=4 mice/condition). Scale bar=5 μm. (B) Co-incubation of normoxia-treated HPAECs with msAb-N9-P1 and msAb-N9-P2 significantly inhibited TRAP (10 μM)-stimulated platelet-endothelial adhesion. TRAP, thrombin receptor agonist peptide. (C) Significant inhibition of TRAP-stimulated platelet-endothelial adhesion was mediated only by msAb-N9-P2 in hypoxia-treated cells. (D, E) Compared to WT mice, NEDD9^{-/-} mice were resistant to ADP-induced pulmonary arteriolar thrombotic occlusion (arrows) and pulmonary hypertension analyzed by anti-P-Selectin and change in right ventricular systolic pressure (RVSP) by immunofluorescence and cardiac catheterization, respectively. IgG₁ is negative control. Similar effects were observed in WT mice treated with msAb-N9-P2 compared to IgG₁ administered 10 min prior to ADP infusion. Scale bar=5 μm. ADP, adenosine diphosphate. Representative micrographs and hemodynamic tracings are shown. Data are presented as mean±SE.

FIGS. 6A-E. NEDD9 is increased in chronic thromboembolic pulmonary hypertension. (A) Compared to acute pulmonary embolism and deep vein thrombosis (PE/DVT) specimens (N=6), CTEPH-PEA specimens (N=7) were highly fibrotic, and characterized by increased HIF-1α, NEDD9 and P-Selectin-NEDD9 co-localization in platelet aggregates. Data standardized to PECAM-1. Scale bar=40 μm. PEA, pulmonary endarterectomy. (B) Cultured control HPAECs and HPAECs from CTEPH-PEA specimens were analyzed using anti-HIF-1α and anti-NEDD9 (Ab #1) immunoblot (N=3). Dotted line represents unloaded lane on gel that was cropped from image. All samples were run on the same gel. Pt, patient. (C) Anti-NEDD9 immunofluorescence was performed on cultured CTEPH-HPAECs using NEDD9 Ab #1, a monospecific anti-NEDD9 against substrate domain P1 (LYQVPNPQAAPR, SEQ ID NO:3) (msAb-N9-P1), or a monospecific anti-NEDD9 against substrate domain P2 (GPVFSVPVGEIKPQGVYDIPPTK; SEQ ID NO:4) (msAb-N9-P2). (D) Platelet-endothelial adhesion was analyzed in CTEPH-HPAECs and control HPAECs incubated with platelets from healthy donors under basal conditions and following stimulation with TRAP (10 μM) in the presence or absence of msAb-N9-P1 or msAb-N9-P2. Treatment with IgG₁ served as negative control (N=4). (E) Plasma NEDD9 was increased significantly in CTEPH patients (N=27) compared with age- and sex-matched healthy controls (N=7). PEA, pulmonary endarterectomy. Representative micrographs and immunoblots are shown. For (A-D): data are presented as mean±SE. For E: mean, square; median, horizontal line; interquartile range, box distribution; maximum and minimum, y-axis lines. Scale bar=20 μm.

FIG. 7. Pool of NEDD9 peptides isolated from human pulmonary artery endothelial cells by IP/LC-MS. Lysates from untreated human pulmonary artery endothelial cells (HPAECs) were immunoprecipitated (IP) using NEDD9 Ab #1 or NEDD9 Ab #2, run on an SDS-PAGE gel, and subjected to in-gel trypsin digestion. Samples were then analyzed using liquid chromatography-mass spectrometry (LC-MS) for peptide identification, and the amino acid sequences of NEDD9 peptides detected using this method are provided (N=3 HPAEC samples).

FIGS. 8A-B. The effect of siRNA-NEDD9 on NEDD9 expression in human pulmonary artery endothelial cells (HPAECs). (A) HPAECs were transfected with vehicle (Lipofectamine) control, scrambled (negative) control siRNA, or NEDD9 siRNA (si-NEDD9) (20-60 nM). Cells were collected 24 hr or 48 hr following transfection and analyzed using anti-NEDD9 (Ab #1) immunoblot (N=3) or (B) anti-NEDD9 immunofluorescence (N=3). IgG₁ antibody was used as negative control. Data are presented as mean±SEM. Representative immunoblots and micrographs are shown. Scale bar=20 m. a.u., arbitrary units.

FIGS. 9A-C. Liquid chromatography-mass spectrometry confirms the amino acid sequences of the NEDD9 P1 and NEDD9 P2 model peptides. Based on our findings in human pulmonary artery endothelial cells, we synthesized two model peptides representing putative NEDD9 binding targets of P-Selectin (NEDD9 P1 and NEDD9 P2) and analyzed the amino acid sequence by liquid chromatography-mass spectrometry. (A) MS1 and (B) MS2 spectra corresponding to the NEDD9 P1 peptide: CFGQQKLYQVPNPQAAPRDT-amide (SEQ ID NO:6)(monoisotopic mass=2259.11 Da; average mass=2260.53 Da; HPLC purity=99.86%). (C) MS1 spectra corresponding to the NEDD9 P2 peptide: CGEIKPQGVYDIPPTKGV-amide (SEQ ID NO:8) (monoisotopic mass=2259.11 Da; average mass=2260.53 Da; HPLC purity=96.25%). HPLC, high performance liquid chromatography.

FIGS. 10A-D. The custom-made monospecific anti-NEDD9 antibodies are specific to NEDD9 with species cross-reactivity. (A,B) Custom-made monospecific antibodies against NEDD9 substrate domain peptide 1 (msAb-N9-P1) and peptide 2 (msAb-N9-P2) were developed. The specificity of msAb-N9-P1 and msAb-N9-P2 was established by immunoblot using recombinant NEDD9 and recombinant p130Cas, which like NEDD9 is also a Cas protein and shares up to 92% amino acid similarity with NEDD9 in specific domains (N=3). (C) Homology for NEDD9 at P1 and P2 between Homo sapiens and Mus musculus. (D) Cross-species detection for human and mouse NEDD9 analyzed by anti-msAb-N9-P1 and anti-msAb-N9-P2 immunoblot (N=3). msAb-N9-P1, monospecific anti-NEDD9 antibody against the NEDD9 P1 peptide; msAb-N9-P2, monospecific anti-NEDD9 antibody against the NEDD9 P2 peptide. Representative immunoblots are shown. a.u., arbitrary units.

FIGS. 11A-B. Inhibition of NEDD9-P-Selectin complex formation by msAb-N9-P1 and msAb-N9-P2 in a cell-free system in vitro. (A) Recombinant NEDD9 (5 ng) and P-Selectin (5 ng) were incubated for 30 min in solution with the following treatments: msAb-N9-P1 (10-20 μM), msAb-N9-P2 (10-20 μM), or IgG₁ (10 μg) as control. (B) The effect of treatment on the NEDD9-P-Selectin complex was analyzed by anti-P-Selectin immunoprecipitation (IP) followed by anti-NEDD9 immunoblot (IB) using NEDD9 Ab #1 (N=3). msAb-N9-P1/2, incubation with antibodies alone as negative control; PBS, phosphate buffered saline alone as negative control. Data are presented as mean±SEM. Representative immunoblots are shown. a.u., arbitrary units.

FIGS. 12A-D. NEDD9 correlates with P-Selectin and HIF-1α in DVT/PE and CTEPH endarterectomy samples ex vivo. Levels of NEDD9, P-Selectin, and hypoxia inducible factor (HIF)-1a were quantified by immunofluorescence using samples from patients with deep vein thrombosis/acute pulmonary embolism (DVT/PE) (N=6) or CTEPH endarterectomy (N=7). Linear regression analyses were then performed comparing NEDD9 with (A) P-Selectin and (B) HIF-1α. (C, D) The relationship between NEDD9, P-Selectin and HIF-1α is provided using a 3-dimensional (xyz axis) surface plot, provided in two orthogonal views. a.u., arbitrary units.

FIGS. 13A-B. Unstimulated platelets from patients with CTEPH demonstrate increased activity compared to healthy donor controls. (A) Compared to control human pulmonary artery endothelial cells (HPAECs), CTEPH-HPAECs demonstrate increased platelet-endothelial adhesion under basal conditions (N=3). Data in the bar graph compare results for HPAECs and CTEPH-HPAECs obtained from the same experimental method that did not include both conditions in the same assay run, indicated by the break in x-axis. (B) Platelets from patients CTEPH were analyzed by flow cytometry in the absence of exogenous platelet stimulators (N=3). The percentage of platelets expressing P-Selectin and IgG₁ as a measure of platelet activation and negative control, respectively, are reported in the gating plots. Data in the bar graph compare the percentage of P-selectin-expressing platelets in CTEPH vs. normal controls as reported in FIG. 3A, indicated by break in the x-axis. Representative flow cytometry plots are shown. CTEPH, chronic thromboembolic pulmonary hypertension. Data are presented as mean±SE.

FIG. 14. Timeline, treatment time point, and expected time required for completion for each PVTE animal model. d, day; ADP, adenosine diphosphate; SU-5416, Sugen-5416; mAb-N9, monoclonal antibody against thrombogenic NEDD9.

### DETAILED DESCRIPTION

Chronic thromboembolic pulmonary hypertension (CTEPH) is a distinct disease defined, in part, by increased platelet-endothelial adhesion resulting in organized thromboembolism, vascular fibrosis, and early mortality.¹ Pulmonary endarterectomy (PEA) is the definitive treatment for CTEPH, but is associated with significant morbidity and may be inappropriate or unsuccessful in up to one-third of patients.^{2,3} The single drug therapy approved for use in CTEPH clinically is repurposed from pulmonary arterial hypertension, which is distinct in pathogenesis and epidemiology. Thus, identifying CTEPH-specific pathobiological mechanism(s) is likely to advance disease-modifying treatments for patients.

Data from observational studies and case reports propose an association between CTEPH prevalence and rare variants in genes encoding coagulation proteins (or co-factors),⁴ or that affected patients harbor non-specific platelet or coagulation cascade abnormalities.^{5,6} However, the CTEPH pathophenotype is complex, and includes pulmonary endothelial dysfunction, vascular hypoxia, and propagation of thrombotic remodeling that implies dysregulated cell-cell interactions.

Developing a PVTE/CTEPH therapy that is selective to the lung is anticipated to provide a superior therapeutic advantage compared to the current standard of care by enhancing its efficacy and safety profile. In patients diagnosed with PVTE, the current standard of care treatment is anticoagulation, systemic thrombolysis, catheter-directed thrombolysis, or surgical clot resection. Anticoagulant drugs affect general coagulation cascade proteins or co-factors to limit clot propagation, but therapeutic efficacy hinges on the endogenous fibrinolytic system for clot resolution. Generally, thrombolytics activate plasminogen, the zymogen of the proteolytic enzyme plasmin. Increased plasmin catabolizes cross-links between fibrin molecules to dissolve clots. These drugs may be administered using an intravenous or central catheter. However, thrombolytic and anticoagulant drugs are not pulmonary circulation-specific, and do not target PVTE-specific molecular mechanisms. Thus, these therapies are associated with incomplete treatment effect and unacceptable rates of major/fatal bleeding events. Pulmonary thromboendarterectomy is the mainstay treatment for CTEPH, but is unsuccessful in 30% of patients and is associated with increased risk of major post-operative complications (e.g., post-operative infection, neurological complications, and mortality in 20%, 13%, and ˜5% of patients, respectively; see Delcroix et al. Circulation. 2016 Mar. 1; 133(9):859-7). Furthermore, ˜40% of patients report impaired quality of life and functional status 1 month after acute PE (Kahn et al. J Thromb Haemost 2008; 6: 1105-12), indicating that standard-of-care therapy is ineffective in the intermediate- and long-term.

The present inventors hypothesized that upregulation of NEDD9 in HPAECs by hypoxia might affect platelet-endothelial adhesion and could be an important prothrombotic mechanism underlying CTEPH. As demonstrated herein, HIF-1α-dependent upregulation of NEDD9 in HPAECs promoted the formation of a previously unrecognized protein-protein complex between NEDD9 and P-Selectin, which in turn, modulated platelet-HPAEC adhesion in vitro and pulmonary arterial thrombosis in vivo. Platelet-rich PEA specimens, plasma, and HPAECs from CTEPH patients expressed increased NEDD9, providing a disease correlate to these findings. A specific peptide in the tyrosine-rich substrate domain of NEDD9 that is accessible on the extracellular HPAEC plasma membrane was sequenced and used to develop anti-NEDD9 antibodies (including the monospecific msAb-N9-P2). Inhibition of platelet adhesion to CTEPH-HPAECs ex vivo and pulmonary hypertension in mice stimulated with ADP by msAb-N9-P2 proved that NEDD9 is a modifiable target by which to prevent occlusive pulmonary thrombosis. Collectively, these findings indicated that NEDD9 bioactivity is at a convergence point of hypoxia signal transduction and endothelial dysfunction with important implications for the pathogenesis of CTEPH.

Impaired fibrinolysis following a luminal PE has been proposed to explain CTEPH based on findings from epidemiological studies and case reports as well as empiric data implicating diminished plasminogen activator inhibitor activity, increased bioavailable thrombin activatable fibrinolysis inhibitor, and polymorphisms in the gene coding fibrin in affected patients.^{4,25-28} Hypercoagulability may also predispose to CTEPH, as elevated levels of factor VIII are reported in patient cohort studies.²⁹ However, these risk factors overlap with coronary and cerebral thromboembolic disorders, and, therefore, do not necessarily provide unique insight into the pathogenesis of CTEPH or other pulmonary vascular diseases. The present results imply that hypoxia upregulates NEDD9 in HPAECs, which was not reproduced in coronary or cerebral microvascular endothelial cells, and that NEDD9 bioactivity may drive divergence in the pathobiology of CTEPH from PE/DVT. Leveraging cell-specific responses to hypoxia has important implications on drug development in CTEPH. Findings from this study, for example, establish a framework for pulmonary circulatory-specific pharmacotherapies: the principal ligand for msAb-N9 was not increased by hypoxia in HPAECs, but this was not the case in off-target cell types.

The protein docking function of NEDD9 has been reported previously, including in cancer metastasis via cell-cell interactions involving focal adhesion kinase,³⁰ and in vascular fibrosis by virtue of its association with SMAD^{3,11} among other processes. This work expands the gamut of NEDD9 binding targets to include P-Selectin, which to the present inventors' knowledge has not been reported previously. Early work focusing on P-Selectin showed strong affinity at Tyr¹⁴⁸ in the extracellular domain of its counter receptor, P-Selectin Glygcoprotein Ligand-1.¹⁸ P-Selectin is an established mediator of pulmonary arterial thrombosis³¹ with relevance to pulmonary vascular disease,³² and the LC-MS data herein identified the NEDD9 tyrosine rich substrate domain in HPAEC plasma membrane isolates. A combination of methods, including microscale thermophoresis, was used to definitively establish the formation of a P-Selectin-NEDD9 complex. Furthermore, the K_{D} of this association was in the range reported for other clinically relevant platelet-endothelial interactions, such as Glycoprotein IIb/IIIa-von Willebrand Factor,³³ providing important biological and pharmacological context to the P-Selectin-NEDD9 interaction.

Prior reports exploring the relationship between hypoxia and HIF-1α-dependent upregulation of NEDD9 have focused on the tumor microenvironment.^{8,34} Determining that NEDD9 is a HIF-1α target in HPAECs, however, has several unique implications to pulmonary thromboembolic disease. First, vascular remodeling in CTEPH correlates positively with persistent hypoxemia following PEA,³⁵ and PEA specimens express a high population of HIF-1α positive cells that may persist for years following the sentinel event (i.e., acute PE).¹⁰ Thus, chronic overactivation of HIF-1α-NEDD9 signaling may provide mechanistic insights to the phenotype transition from luminal PE to CTEPH. Second, emphasizing PAEC-hypoxia signaling pathways is likely to elucidate the mechanisms that switch the endothelium to a prothrombotic organ. Doing so may widen the range of potential therapeutic targets in CTEPH beyond coagulation cascade intermediaries alone: endothelial dysfunction defined in this study by increased N9-P2 emerged as a novel and modifiable molecular target by which to restore the normal anti-thrombotic endothelium and limit platelet adhesion.

This study identifies NEDD9 as a heretofore unrecognized mediator of platelet-endothelial adhesion, and expands the understanding of protein-protein interactions involved in the pathogenesis of cardiovascular disease. NEDD9-mediated pulmonary arterial thrombosis is modifiable pharmacologically, which was accomplished through the development of an anti-NEDD9 antibody targeting the extracellular peptide that ligands with P-Selectin. Overall, these data illustrate an innovative and clinically relevant molecular mechanism with direct relevance to the pathogenesis of CTEPH and other diseases characterized by pulmonary vascular thrombotic events.

Anti-NEDD9 Antibodies

Provided herein are anti-NEDD9 antibodies that bind to NEED9, in or near a NEDD9 substrate domain, e.g., a substrate domain that comprises one or more YxxP motifs, e.g., a tyrosine rich substrate domain that is accessible on the extracellular HPAEC plasma membrane. In some instances, the antibodies described herein bind to an epitope in or near a NEDD9 substrate domain, e.g., within one of the following sequences: NEDD9 AA 75-125: EQPASG LMQQTFGQQK LYQVPNPQAA PRDTIYQVPP SYQNQGIYQV PTGHG (SEQ ID NO:1); or NEDD9 AA 175-225: DVYDIP PSHTTQGVYD IPPSSAKGPV FSVPVGEIKP QGVYDIPPTK GVYAI (SEQ ID NO:2). As used herein, “near” a substrate domain means within 50 amino acids, e.g., within 40, 30, 25, 20, 10, or 5 amino acids of a 5′ or 3′ end of a substrate domain sequence as described herein. In some instances, the antibodies described herein bind to or near an epitope in NEDD9 substrate domain, e.g., within K.LYQVPNPQAAPR.D (SEQ ID NO:9) or K.GPVFSVPVGEIKPQGVYDIPPTK.G (SEQ ID NO:10). An exemplary full sequence of human NEDD9 protein is in GenBank at NP_006394.1.

In some instances, the antibodies provided herein block the interaction between NEDD9 protein and P-selectin. The antibodies provided herein may reduce the binding affinity of a protein-protein complex between NEDD9 and P-Selectin, or block formation of the P-Selectin-NEDD9 complex. In some instances, the antibodies provided herein bind to the substrate domain of a wild type NEDD9 protein. In some instances, the antibodies described herein reduce PVTE formation and/or platelet-endothelial adhesion.

In some instances, the antibodies provided herein bind to an amino acid sequence in NEDD9 that comprises or consists of K.LYQVPNPQAAPR.D (SEQ ID NO:9) or K.GPVFSVPVGEIKPQGVYDIPPTK.G (SEQ ID NO: 10). In some instances, the amino acid sequence K.LYQVPNPQAAPR.D (SEQ ID NO:9) comprises or consists of an epitope for the antibodies provided herein. In some instances, the amino acid sequence K.GPVFSVPVGEIKPQGVYDIPPTK.G (SEQ ID NO:10) comprises or consists of an epitope for the antibodies provided herein.

Variants of these sequences can also be used, e.g., that are at least 80%, 85%, 90%, or 95% identical to these sequences. Calculations of “identity” between two sequences can be performed as follows. The sequences are aligned for optimal comparison purposes (e.g., gaps can be introduced in one or both of a first and a second nucleic acid sequence for optimal alignment and non-identical sequences can be disregarded for comparison purposes). The length of a sequence aligned for comparison purposes is at least 70% (e.g., at least 80%, 90% or 100%) of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In some embodiments, the percent identity between two nucleotide sequences is determined using the Needleman and Wunsch ((1970) J. Mol. Biol. 48:444-453) algorithm, which has been incorporated into the GAP program in the GCG software package (available at gcg.com), using either a Blossum 62 matrix, a PAM250 matrix, a NWSgapdna.CMP matrix. In some embodiments, the percent identity between two amino acid or nucleotide sequences can be determined using the algorithm of E. Meyers and W. Miller ((1989) CABIOS, 4:11-17) which has been incorporated into the ALIGN program (version 2.0), using a PAM120 weight residue table, a gap length penalty of 12 and a gap penalty of 4.

In some embodiments, an antibody or NEDD9-binding fragment thereof described herein demonstrates the binding characteristics and/or biological properties as outlined for the antibodies illustrated in the Examples section below.

Usage of the term “antibody” in this disclosure is meant to cover a whole antibody (as opposed to a minibody, nanobody or antibody fragment), a bispecific antibody, a tertravalent antibody, a multispecific antibody, a minibody, a nanobody, and antibody fragments. In some instances, the anti-NEDD9 antibody of this disclosure is a whole antibody. In certain instances, the heavy chain constant region of the anti-NEDD9 antibody is a human IgG1, human IgG2, human IgG3, or human IgG4 constant region. In certain instances, the light constant region is a human kappa constant region. In other instances, the light constant region is a human lambda constant region. In some instances, the antibodies of this disclosure are designed to have low effector functionality (e.g., by Fc modifications such as N297Q, T299A, etc. See, also, Wang, X., Mathieu, M. & Brezski, R. J. Protein Cell (2018) 9: 63. doi.org/10.1007/s13238-017-0473-8 (incorporated by reference herein)). In some cases, the Fc moiety of the antibody is a hIgG1 Fc, a hIgG2 Fc, a hIgG3 Fc, a hIgG4 Fc, a hIgG1agly Fc, a hIgG2 SAA Fc, a hIgG4(S228P) Fc, or a hIgG4(S228P)/G1 agly Fc (in this format—that minimizes effector function—the CH1 and CH2 domains are IgG4 with a ‘fixed’ hinge (S228P) and is aglycosylated. The CH3 domain is hIgG1, or a hIgG4(S228P) agly Fc). In one case, the antibody has one of the following three scaffolds with reduced effector function: hIgG1 agly (N297Q); hIgG2 SAA (see, Vafa et al. Methods, 65(1):114-26 (2014); and hIgG4P/G1 agly (see, US 2012/0100140 A1).

Antibody Fragments

Antibody fragments (e.g., Fab, Fab′, F(ab′)2, Facb, and Fv) can be prepared by proteolytic digestion of intact antibodies. For example, antibody fragments can be obtained by treating the whole antibody with an enzyme such as papain, pepsin, or plasmin. Papain digestion of whole antibodies produces F(ab)2 or Fab fragments; pepsin digestion of whole antibodies yields F(ab′)2 or Fab′; and plasmin digestion of whole antibodies yields Facb fragments.

Alternatively, antibody fragments can be produced recombinantly. For example, nucleic acids encoding the antibody fragments of interest can be constructed, introduced into an expression vector, and expressed in suitable host cells. See, e.g., Co, M. S. et al., J. Immunol., 152:2968-2976 (1994); Better, M. and Horwitz, A. H., Methods in Enzymology, 178:476-496 (1989); Pluckthun, A. and Skerra, A., Methods in Enzymology, 178:476-496 (1989); Lamoyi, E., Methods in Enzymology, 121:652-663 (1989); Rousseaux, J. et al., Methods in Enzymology, (1989) 121:663-669 (1989); and Bird, R. E. et al., TIBTECH, 9:132-137 (1991)). Antibody fragments can be expressed in and secreted from E. coli, thus allowing the facile production of large amounts of these fragments. Antibody fragments can be isolated from the antibody phage libraries. Alternatively, Fab′-SH fragments can be directly recovered from E. coli and chemically coupled to form F(ab)2 fragments (Carter et al., Bio Technology, 10:163-167 (1992)). According to another approach, F(ab′)2 fragments can be isolated directly from recombinant host cell culture. Fab and F(ab′) 2 fragment with increased in vivo half-life comprising a salvage receptor binding epitope residues are described in U.S. Pat. No. 5,869,046.

Conjugated Antibodies

The antibodies disclosed herein can be conjugated antibodies that are bound to various molecules including macromolecular substances such as polymers (e.g., polyethylene glycol (PEG), polyethylenimine (PEI) modified with PEG (PEI-PEG), polyglutamic acid (PGA) (N-(2-Hydroxypropyl) methacrylamide (HPMA) copolymers), hyaluronic acid, radioactive materials (e.g. ⁹⁰Y, ¹³¹I), fluorescent substances, luminescent substances, haptens, enzymes, metal chelates, and drugs.

In some embodiments, the antibodies described herein are modified with a moiety that improves its stabilization and/or retention in circulation, e.g., in blood, serum, or other tissues, including the brain, e.g., by at least 1.5, 2, 5, 10, 15, 20, 25, 30, 40, or 50-fold. For example, the antibodies described herein can be associated with (e.g., conjugated to) a polymer, e.g., a substantially non-antigenic polymer, such as a polyalkylene oxide or a polyethylene oxide. Suitable polymers will vary substantially by weight. Polymers having molecular number average weights ranging from about 200 to about 35,000 Daltons (or about 1,000 to about 15,000, and 2,000 to about 12,500) can be used. For example, the antibodies described herein can be conjugated to a water soluble polymer, e.g., a hydrophilic polyvinyl polymer, e.g., polyvinylalcohol or polyvinylpyrrolidone. Examples of such polymers include polyalkylene oxide homopolymers such as polyethylene glycol (PEG) or polypropylene glycols, polyoxyethylenated polyols, copolymers thereof and block copolymers thereof, provided that the water solubility of the block copolymers is maintained. Additional useful polymers include polyoxyalkylenes such as polyoxyethylene, polyoxypropylene, and block copolymers of polyoxyethylene and polyoxypropylene; polymethacrylates; carbomers; and branched or unbranched polysaccharides.

The above-described conjugated antibodies can be prepared by performing chemical modifications on the antibodies or the lower molecular weight forms thereof described herein. Methods for modifying antibodies are well known in the art (e.g., U.S. Pat. Nos. 5,057,313 and 5,156,840).

The anti-NEDD9 antibodies can be in the form of full length (or whole) antibodies, or in the form of low molecular weight forms (e.g., biologically active antigen-binding antibody fragments or minibodies) of the anti-NEDD9 antibodies, e.g., Fab, Fab′, F(ab′)₂, Fv, Fd, dAb, scFv, and sc(Fv)2. Other anti-NEDD9 antibodies encompassed by this disclosure include single domain antibody (sdAb) containing a single variable chain such as, VH or VL, or a biologically active fragment thereof. See, e.g., Moller et al., J. Biol. Chem., 285(49): 38348-38361 (2010); Harmsen et al., Appl. Microbiol. Biotechnol., 77(1):13-22 (2007); U.S. 2005/0079574 and Davies et al. (1996) Protein Eng., 9(6):531-7. Like a whole antibody, a sdAb is able to bind selectively to a specific antigen (e.g., NEDD9). With a molecular weight of only 12-15 kDa, sdAbs are much smaller than common antibodies and even smaller than Fab fragments and single-chain variable fragments.

Nucleic Acids, Vector, Host Cells

This disclosure also features nucleic acids encoding the antibodies disclosed herein. In some instances, the nucleic acids described herein include a nucleic acid encoding the Fc region of a human antibody (e.g., human IgG1, IgG2, IgG3, or IgG4). In certain instances, the nucleic acids include a nucleic acid encoding the Fc region of a human antibody that has been modified to reduce or eliminate effector function (e.g., a N297Q or T299A substitution in a human IgG1 Fc region (numbering according to EU numbering)). In some cases, the nucleic acids include a nucleic acid encoding an Fc moiety that is a hIgG1 Fc, a hIgG2 Fc, a hIgG3 Fc, a hIgG4 Fc, a hIgG1agly Fc, a hIgG2 SAA Fc, a hIgG4(S228P) Fc, or a hIgG4(S228P)/G1 agly Fc.

Also disclosed herein are vectors (e.g. expression vectors) containing any of the nucleic acids described above.

Furthermore, this disclosure relates to host cells (e.g. bacterial cells, yeast cells, insect cells, or mammalian cells) containing the vector(s) or the nucleic acid(s) described above.

Methods of Obtaining Anti-NEDD9 Antibodies

Also provided herein are methods for making anti-NEDD9 antibodies useful in the present methods. General methods for making antibodies, e.g., monospecific, polyclonal, or monoclonal antibodies, are known in the art. For monoclonal antibodies, the process involves obtaining antibody-secreting immune cells (lymphocytes) from the spleen of a mammal (e.g., mouse) that has been previously immunized with the antigen of interest (e.g., a peptide antigen as described herein) either in vivo or in vitro. The antibody-secreting lymphocytes are then fused with myeloma cells or transformed cells that are capable of replicating indefinitely in cell culture, thereby producing an immortal, immunoglobulin-secreting cell line. The resulting fused cells, or hybridomas, are cultured, and the resulting colonies screened for the production of the desired monoclonal antibodies. Colonies producing such antibodies are cloned, and grown either in vivo or in vitro to produce large quantities of antibody. A description of the theoretical basis and practical methodology of fusing such cells is set forth in Kohler and Milstein, Nature 256:495 (1975).

Mammalian lymphocytes are immunized by in vivo immunization of the animal (e.g., a mouse) with a peptide antigen, e.g., a peptide antigen that is at least 80%, 85%, 90%, or 95% identical to K.LYQVPNPQAAPR.D (SEQ ID NO:9) or K.GPVFSVPVGEIKPQGVYDIPPTK.G (SEQ ID NO:10), optionally with one or more substitutions or deletions, e.g., of up to 20% of the residues. For example, the methods can include immunizing the animal with a peptide comprising a sequence that is at least 80% identical to at least 10 consecutive amino acids from: (i) the NEDD9 substrate domain P1, e.g., a peptide comprising LYQVPNPQAAPR (SEQ ID NO:3) or CFGQQKLYQVPNPQAAPRDT-amide (SEQ ID NO:6) (the CFGQQK (SEQ ID NO:11) being added for stability), or (ii) NEDD9 substrate domain P2, e.g., a peptide comprising GEIKPQGVYDIPPTKGV (SEQ ID NO:7) or CGEIKPQGVYDIPPTKGV-amide (SEQ ID NO:8). Such immunizations are repeated as necessary at intervals of up to several weeks to obtain a sufficient titer of antibodies. Following the last antigen boost, the animals are sacrificed and spleen cells removed.

Fusion with mammalian myeloma cells or other fusion partners capable of replicating indefinitely in cell culture is effected by known techniques, for example, using polyethylene glycol (“PEG”) or other fusing agents (See Milstein and Kohler, Eur. J. Immunol. 6:511 (1976), which is hereby incorporated by reference). This immortal cell line, which is preferably murine, but can also be derived from cells of other mammalian species, including but not limited to rats and humans, is selected to be deficient in enzymes necessary for the utilization of certain nutrients, to be capable of rapid growth, and to have good fusion capability. Many such cell lines are known to those skilled in the art, and others are regularly described.

Procedures for raising polyclonal antibodies are also known. Typically, such antibodies can be raised by administering the protein or polypeptide of the present invention subcutaneously to New Zealand white rabbits that have first been bled to obtain pre-immune serum. The antigens can be injected, e.g., at a total volume of 100 μl per site at six different sites. Each injected material will contain synthetic surfactant adjuvant pluronic polyols, or pulverized acrylamide gel containing the protein or polypeptide after SDS-polyacrylamide gel electrophoresis. The rabbits are then bled two weeks after the first injection and periodically boosted with the same antigen three times every six weeks. A sample of serum is then collected 10 days after each boost. Polyclonal antibodies are then recovered from the serum by affinity chromatography using the corresponding antigen to capture the antibody. Ultimately, the rabbits are euthanized, e.g., with pentobarbital 150 mg/Kg IV. This and other procedures for raising polyclonal antibodies are disclosed in E. Harlow, et. al., editors, Antibodies: A Laboratory Manual (1988).

The methods described herein can comprise any one of the step(s) of producing a chimeric antibody, humanized antibody, single-chain antibody, Fab-fragment, bi-specific antibody, fusion antibody, labeled antibody or an analog of any one of those. Corresponding methods are known to the person skilled in the art and are described, e.g., in Harlow and Lane “Antibodies, A Laboratory Manual”, CSH Press, Cold Spring Harbor (1988). When derivatives of said antibodies are obtained by the phage display technique, surface plasmon resonance as employed in the BIAcore system can be used to increase the efficiency of phage antibodies which bind to the same epitope as that of any one of the antibodies described herein (Schier, Human Antibodies Hybridomas 7 (1996), 97-105; Malmborg, J. Immunol. Methods 183 (1995), 7-13). The production of chimeric antibodies is described, for example, in international application WO89/09622. Methods for the production of humanized antibodies are described in, e.g., European application EP-A1 0 239 400 and international application WO90/07861. A further source of antibodies to be utilized in accordance with the present invention are so-called xenogeneic antibodies. The general principle for the production of xenogeneic antibodies such as human-like antibodies in mice is described in, e.g., international applications WO91/10741, WO94/02602, WO96/34096 and WO 96/33735. As discussed above, the antibody described herein may exist in a variety of forms besides complete antibodies; including, for example, Fv, Fab and F(ab)2, as well as in single chains; see e.g. international application WO88/09344.

Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow et al., Antibodies: A Laboratory Manual, Cold Spring Harbor Laboratory Press, 2nd ed. (1988); Hammerling et al., in: Monoclonal Antibodies and T-Cell Hybridomas Elsevier, N.Y., 563-681 (1981), said references incorporated by reference in their entireties. The term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced. Thus, the term “monoclonal antibody” is not limited to antibodies produced through hybridoma technology.

In the known hybridoma process (Kohler et al., Nature 256 (1975), 495) the relatively short-lived, or mortal, lymphocytes from a mammal, e.g., B cells derived from a murine subject as described herein, are fused with an immortal tumor cell line (e.g., a myeloma cell line), thus, producing hybrid cells or “hybridomas” which are both immortal and capable of producing the genetically coded antibody of the B cell. The resulting hybrids are segregated into single genetic strains by selection, dilution, and re-growth with each individual strain comprising specific genes for the formation of a single antibody. They produce antibodies, which are homogeneous against a desired antigen and, in reference to their pure genetic parentage, are termed “monoclonal”.

Hybridoma cells thus prepared are seeded and grown in a suitable culture medium that contain one or more substances that inhibit the growth or survival of the unfused, parental myeloma cells. Those skilled in the art will appreciate that reagents, cell lines and media for the formation, selection and growth of hybridomas are commercially available from a number of sources and standardized protocols are well established. Generally, culture medium in which the hybridoma cells are growing is assayed for production of monoclonal antibodies against the desired antigen. The binding specificity of the monoclonal antibodies produced by hybridoma cells is determined by in vitro assays such as immunoprecipitation, radioimmunoassay (RIA) or enzyme-linked immunoabsorbent assay (ELISA) as described herein. After hybridoma cells are identified that produce antibodies of the desired specificity, affinity and/or activity, the clones may be subcloned by limiting dilution procedures and grown by standard methods; see, e.g., Goding, Monoclonal Antibodies: Principles and Practice, Academic Press, pp 59-103 (1986). It will further be appreciated that the monoclonal antibodies secreted by the subclones may be separated from culture medium, ascites fluid or serum by conventional purification procedures such as, for example, protein-A, hydroxylapatite chromatography, gel electrophoresis, dialysis or affinity chromatography.

In another embodiment, lymphocytes can be selected by micromanipulation and the variable genes isolated. For example, peripheral blood mononuclear cells can be isolated from an immunized or naturally immune mammal, e.g., a human, and cultured for about 7 days in vitro. The cultures can be screened for specific immunoglobulins that meet the screening criteria. Cells from positive wells can be isolated. Individual Ig-producing B cells can be isolated by FACS or by identifying them in a complement-mediated hemolytic plaque assay. Ig-producing B cells can be micromanipulated into a tube and the VH and VL genes can be amplified using, e.g., RT-PCR. The VH and VL genes can be cloned into an antibody expression vector and transfected into cells (e.g., eukaryotic or prokaryotic cells) for expression.

Alternatively, antibody-producing cell lines may be selected and cultured using techniques well known to the skilled artisan. Such techniques are described in a variety of laboratory manuals and primary publications. In this respect, techniques suitable for use in the invention as described below are described in Current Protocols in Immunology, Coligan et al., Eds., Green Publishing Associates and Wiley-Interscience, John Wiley and Sons, New York (1991) which is herein incorporated by reference in its entirety, including supplements.

Methods of generating variants (e.g., comprising amino acid substitutions) of any of the anti-NEDD9 antibodies are well known in the art. These methods include, but are not limited to, preparation by site-directed (or oligonucleotide-mediated) mutagenesis, PCR mutagenesis, and cassette mutagenesis of a prepared DNA molecule encoding the antibody or any portion thereof (e.g., a framework region, a CDR, a constant region). Site-directed mutagenesis is well known in the art (see, e.g., Carter et al., Nucl. Acids Res., 13:4431-4443 (1985) and Kunkel et al., Proc. Natl. Acad. Sci. USA, 82:488 (1987)). PCR mutagenesis is also suitable for making amino acid sequence variants of the starting polypeptide. See Higuchi, in PCR Protocols, pp. 177-183 (Academic Press, 1990); and Vallette et al., Nucl. Acids Res. 17:723-733 (1989). Another method for preparing sequence variants, cassette mutagenesis, is based on the technique described by Wells et al., Gene, 34:315-323 (1985).

See, e.g., US20180371070, US20170029525, and US20180346553.

Methods of Treatment

The methods described herein include methods for the treatment of disorders associated with pulmonary vascular thromboembolism (PVTE). In some embodiments, the disorder is luminal pulmonary embolism (PE), cancer-associated PE, pulmonary arterial hypertension (PAH), and chronic thromboembolic pulmonary hypertension (CTEPH). Generally, the methods include administering a therapeutically effective amount of an NEDD9 antibody as described herein, to a subject who is in need of, or who has been determined to be in need of, such treatment. Methods for identifying such subjects are known in the art, e.g., using ventilation/perfusion (V/Q) scintigraphy; pulmonary angiography; Dual-Energy Computed Tomography angiography (DECT); and/or Computed Tomography angiography (CTA) (see, e.g., Maron et al., JAMA Cardiol. 2016 Dec. 1; 1(9): 1056-1065; Gopalan et al., European Respiratory Review 2017 26: 160108; Kharat et al., Thromb Res. 2018 March; 163:260-265; Corrigan et al., Clin Exp Emerg Med. 2016 September; 3(3): 117-125; van Beek et al., Continuing Education in Anaesthesia Critical Care & Pain, August 2009, 9(4): 119-124; Sakuma et al., Circ J. 2005 September; 69(9):1009-15.

As used in this context, to “treat” means to ameliorate at least one symptom of the disorder associated with PVTE. Often, PVTE results in pulmonary hypertension or embolism; thus, a treatment can result in treatment of, or a reduction in risk or severity of, pulmonary hypertension or embolism.

In some embodiments, the methods include administration of a second treatment modality for the disorder associated with PVTE, e.g., anticoagulation therapy (e.g., warfarin or direct oral anticoagulants, e.g., apixaban (Eliquis®), betrixaban (BevyxXa®), dabigatran (Pradaxa®), edoxaban (Savaysa®) and rivaroxaban (Xarelto®)); a stimulator of soluble guanylate cyclase (sGC), e.g., Riociguat (Adempas®); systemic thrombolysis, catheter-directed thrombolysis, or surgical clot resection (Surgical thrombectomy). See, e.g., Kabrhel et al., Acad Emerg Med. 2017 October; 24(10):1235-1243. Medical therapies for pulmonary arterial hypertension can also be administered, e.g., vasodilators and anti-proliferative agents, e.g., Epoprostenol (Flolan), Epoprostenol (Veletri), Treprostinil (Remodulin), Iloprost (Ventavis), Treprostinil (Tyvaso), Bosentan (Tracleer), Ambrisentan (Letairis), Sildenafil (Revatio), or Tadalafil (Adcirca); Calcium Channel Blockers; Blood Thinners; Diuretics; Digoxin (Lanoxin); or Oxygen. See, e.g., Pulido et al., Heart Failure Reviews May 2016, Volume 21, Issue 3, pp 273-283.

In some embodiments, the methods include long-term oral administration of mAb-N9 in the sub-acute management phase of PE and cancer-associated PE (e.g., ambulatory care post-hospital discharge, ≥6 months), as well as long-term (e.g., indefinite) treatment of PAH and CTEPH. In cancer patients at risk for PVTE the present methods can be used for reduction of risk of cancer-associated PE. Such subjects include subjects with metastatic disease at the time of presentation and who have fast growing, biologically aggressive cancers associated with a poor prognosis; subjects with haematological, pancreatic, ovarian, or brain cancer; or subjects who are being treated with therapy that increases the risk of PVTE, e.g., Fluorinated pyrimidines (e.g., 5-fluorouracil (5-Fu), capecitabine, tegafur-uracil, S1); Cisplatin; L-asparaginase; Tamoxifen; Dexamethasone; Erythropoiesis-stimulating agents; or ImiDs (e.g., thalidomide, lenalidomide, etc.). See, e.g., Lee and Levine, Circulation. 2003; 107:I-17-I-21; Best Pract Res Clin Haematol. 2009 March; 22(1): 9-23; Khalil et al., World J Surg Oncol. 2015; 13: 204).

Pharmaceutical Compositions and Methods of Administration

The methods described herein include the use of pharmaceutical compositions comprising an anti-NEDD9 antibody as described herein as an active ingredient.

Pharmaceutical compositions typically include a pharmaceutically acceptable carrier. As used herein the language “pharmaceutically acceptable carrier” includes saline, solvents, dispersion media, coatings, antibacterial and antifungal agents, isotonic and absorption delaying agents, and the like, compatible with pharmaceutical administration. Supplementary active compounds can be administered separately or can be incorporated into the compositions, e.g., anticoagulation therapeutics (e.g., warfarin or direct oral anticoagulants, e.g., apixaban (Eliquis®), betrixaban (BevyxXa®), dabigatran (Pradaxa®), edoxaban (Savaysa®) and rivaroxaban (Xarelto®); a stimulator of soluble guanylate cyclase (sGC), e.g., Riociguat (Adempas®); vasodilators and anti-proliferative agents, e.g., Epoprostenol (Flolan), Epoprostenol (Veletri), Treprostinil (Remodulin), Iloprost (Ventavis), Treprostinil (Tyvaso), Bosentan (Tracleer), Ambrisentan (Letairis), Sildenafil (Revatio), or Tadalafil (Adcirca); Calcium Channel Blockers; Blood Thinners; Diuretics; Digoxin (Lanoxin); or Oxygen.

Pharmaceutical compositions are typically formulated to be compatible with its intended route of administration. Examples of routes of administration include parenteral, e.g., intravenous, intradermal, subcutaneous, oral (e.g., inhalation), transdermal (topical), transmucosal, and rectal administration.

Methods of formulating suitable pharmaceutical compositions are known in the art, see, e.g., Remington: The Science and Practice of Pharmacy, 21st ed., 2005; and the books in the series Drugs and the Pharmaceutical Sciences: a Series of Textbooks and Monographs (Dekker, NY). For example, solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yield a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds can be delivered in the form of an aerosol spray from a pressured container or dispenser that contains a suitable propellant, e.g., a gas such as carbon dioxide, or a nebulizer. Such methods include those described in U.S. Pat. No. 6,468,798.

Systemic administration of a therapeutic compound as described herein can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The pharmaceutical compositions can also be prepared in the form of suppositories (e.g., with conventional suppository bases such as cocoa butter and other glycerides) or retention enemas for rectal delivery.

In some embodiments, the therapeutic compounds are prepared with carriers that will protect the therapeutic compounds against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques, or obtained commercially, e.g., from Alza Corporation and Nova Pharmaceuticals, Inc. Liposomal suspensions (including liposomes targeted to selected cells with monoclonal antibodies to cellular antigens) can also be used as pharmaceutically acceptable carriers. These can be prepared according to methods known to those skilled in the art, for example, as described in U.S. Pat. No. 4,522,811.

The pharmaceutical compositions can be included in a container, pack, or dispenser together with instructions for administration.

### Examples

The invention is further described in the following examples, which do not limit the scope of the invention described in the claims.

Methods

The following methods were used in the Examples below unless indicated otherwise.

Cell culture and treatments. Details for all cell types and biological reagents used in this study are provided in Tables 2 and 3, respectively. Primary HPAECs (male and female donors), pulmonary artery smooth muscle cells, and coronary artery endothelial cells (all from Lonza) were grown to confluence using EBM-2™ and SmGM-2™, respectively, unless otherwise specified. All medium was supplemented with 5% fetal bovine serum; endothelial and smooth muscle cell medium was also supplemented with a cell type-specific Bulletkit™. C57BL/6 mouse primary PAECs and human brain microvascular endothelial cells (Cell Biologics) were grown to confluence using Cell Biologics Endothelial Cell Medium with Kit™. Cells (passage 3-8) were incubated at 37° C., 5.0% CO₂ and dissociated using 0.5% trypsin/EDTA. In selected experiments, cells were treated with hypoxia (10%, 2%, or 0.2% O₂) using a tightly sealed modular hypoxia chamber incubated at 37° C. for 24 hr, as reported previously.¹²

Platelet-Endothelial Cell Adhesion Assay. Cells were seeded on 96-well opaque-bottom plate (ThermoFisher) and grown to 100% confluence at 37° C., 5.0% CO₂. Human platelets from healthy volunteers were isolated (Partners IRB #2016P001640) and fluorescently labeled with 5-chloromethylfluorescein diacetate (CMFDA) before activation with 10 μM thrombin receptor agonist peptide (TRAP) (Sigma), as described previously.^{13,14} Platelet isolation methods are provided in the on-line Supplement. Platelet number was counted by fluorescence-activated cell sorting and adjusted to 2×10⁸/mL, and then incubated with cell monolayers for 45 min at 37° C., 5.0% CO₂. The total fluorescence [485/535 nm] was measured using a multilabel counter plate reader (Molecular Devices) before and after three serial washes with phosphate buffered saline (PBS). Platelet adhesion (%) was calculated as follows: [remaining fluorescence—blank]÷[total fluorescence—blank]*100.

Human CTEPH Endarteretcomy Samples. Demographic and clinical data for pulmonary endarterectomy (PEA) CTEPH patients are provided in the Tables 1A-C.

Specimens were collected prospectively from CTEPH patients referred for PEA surgery (G.A.A., G.E., R.N.C.) (TR #2016P001640). The PEA specimens were collected in the operating room and divided into proximal and distal sections. Samples were snap frozen in liquid nitrogen or preserved in 10% formalin.

TABLES 1A-C
Thromboembolic specimens were analyzed from patients with
thrombectomy to treat luminal pulmonary embolism/deep vein thrombosis (PE/DVT) or
pulmonary endarterectomy to treat chronic thromboembolic pulmonary hypertension
(CTEPH).
TABLE 1A.

| | | Female | mPAP | PVR | CO | CI |
|---|---|---|---|---|---|---|
| Patient Groups | Age (yr) | (%) | (mm Hg) | (WU) | (L/min) | (L/min/m²) |
| Acute PE/DVT | 56 ± 5.1 | 3 (50) | — | — | — | — |
| (N = 6) | | | | | | |
| CTEPH (N = 7) | 55 ± 6.2 | 4 (57) | 46 ± 4.8 | 8.0 ± 1.3 | 4.4 ± 0.6 | 2.1 ± 0.2 |

TABLE 1B.

| Acute | | | | |
|---|---|---|---|---|
| PE/DVT | Age | | Anatomic Location of | |
| (N = 6) | (yr) | Sex | Specimen | Clinical History |
| 1 | 43 | F | Left brachial vein | Nephrotic syndrome, end-stage |
| | | | thrombus | renal disease, and hypertension |
| 2 | 65 | F | Left lung pulmonary | Deceased; hypertension, asthma, |
| | | | artery embolus | and hypothyroidism |
| 3 | 37 | M | Right lung pulmonary | Deceased; obesity, hypertension, |
| | | | artery embolus | and type 2 diabetes mellitus |
| 4 | 61 | F | Right lung pulmonary | Deceased; Cushing's disease, |
| | | | artery embolus | hypertension, type 2 diabetes |
| | | | | mellitus, obesity, obstructive |
| | | | | sleep apnea, and congestive |
| | | | | heart failure |
| 5 | 67 | M | Left lung pulmonary | Deceased; metastatic prostate |
| | | | artery embolus | cancer and atrial fibrillation |
| 6 | 62 | M | Right pulmonary artery | Surgical embolectomy for |
| | | | embolus | massive PE in baseline obese |
| | | | | individual and active tobacco |
| | | | | use |

TABLE 1C.

| CTEPH | Age | | mPAP | PVR | CO | CI | Pre-PEA |
|---|---|---|---|---|---|---|---|
| (N = 7) | (yr) | Sex | (mmHg) | (WU) | (L/min) | (L/min/m²) | PAH Therapy |
| 1 | 48 | M | 54 | 10.2 | 4.4 | 2.1 | Riociguat |
| 2 | 64 | M | 39 | 5.3 | 3.2 | 1.7 | None |
| 3 | 47 | M | 63 | 14.5 | 3.1 | 1.5 | None |
| 4 | 74 | F | 39 | 6.6 | 4.7 | 2.6 | None |
| 5 | 46 | F | 31 | 7.6 | 2.9 | 1.4 | None |
| 6 | 32 | F | 61 | 6.3 | 7.5 | 3.1 | None |
| 7 | 76 | F | 37 | 5.2 | 4.8 | 2.6 | None |

(1A) Demographic and cardiopulmonary hemodynamic profile of patients. Data were normally distributed and presented as mean ± SE. There was no statistical difference in age between groups (P = 0.94) or sex (P = 0.70) by two sample Student t-test and Chi-square analyses, respectively.
(1B) Individual patient characteristics are provided. M, male; F, female.
(1C) Individual characteristics of CTEPH patients are provided. PEA, pulmonary endarterectomy; PAH, pulmonary arterial hypertension; M, male; F, female. yr, year; mPAP, mean pulmonary artery pressure; PVR, pulmonary vascular resistance; CO, cardiac output; CI, cardiac index; WU, Wood units.

Human CTEPH Pulmonary Artery Endothelial Cells. The CTEPH-HPAECs were isolated at the time of PEA (N=3) (TRB #00082338) using aseptic techniques in a tissue culture hood according to published methods.¹⁵ Briefly, CTEPH thrombi were cut into 1 cm sections and rinsed three times with Hanks' balanced salt solution (Invitrogen). The arteries were then incubated in 10-15 mL of 2 mg/mL type II collagenase (Worthington Biochemical Corporation) in PBS for 20 min at 37° C. and 5% CO₂. After incubation, the samples were massaged with a sterile spatula followed by gentle shaking to detach the endothelial cells into Endothelial Cell Basal Media (Cell Applications #210-500) after addition of a growth supplement kit (Cell Applications, #211-GS) and antibiotic-antimycotic (Invitrogen, #15240062). After removal of the thrombus segments, the sample was centrifuged at 330×g for 7 min at room temperature. The cell pellet was resuspended in the supplemented EC media and seeded on gelatin-coated cell culture plates. The cells were incubated at 37° C., 5% CO₂ with 90% humidity followed by media changes at 24 hr and every 3 d until confluence. Information on CTEPH plasma preparation is provided in the on-line Supplement.

Immunoblotting. Proteins were size-fractionated electrophoretically using SDS-PAGE and transferred to polyvinylidene fluoride membranes according to methods reported previously.³⁸ The membranes were incubated overnight at 4° C. with primary antibodies, outlined in detail in Table 2, incubated with peroxidase-labeled secondary antibody, and visualized using the ECL detection system (Amersham Biosciences). Densitometry was calculated using the ChemiDoc Touch System (Bio-Rad) and standardized to actin. In selected experiments, recombinant NEDD9 and recombinant p130Cas from Origene were used as internal positive controls.

Immunoprecipitation. Magnetic beads (Bio-Rad SureBeads) were resuspended in 100 μl solution (1 mg at 10 mg/mL), magnetized, and serially washed with 1 mL PBS+0.1% Tween 20 (PBS-T). Primary antibody (10 μg) (Table 2) was added to the resuspended beads in a final volume of 200 μl and rotated at room temperature for 10 min. The beads were then magnetized and serially washed with 1 mL PBS-T before incubation with the antigen-containing HPAEC plasma membrane lysate (250-500 μl) plus recombinant P-Selectin (R&D Systems) (0.5-1.0 μg) for 1 hr at room temperature. In the cell-free immunoprecipitation experiment, the antibody-labeled beads were incubated with recombinant NEDD9 (Origene)³⁹ (5 ng) and recombinant P-Selectin (5 ng) in the presence of IgG₁ control, msAb-N9-P1 (10-20 uM), or msAb-N9-P2 (10-20 μM) for 1 hr at room temperature. The beads were then serially washed with 1 mL PBS-T, magnetized, incubated with 40 μl 1× Laemmli buffer (Bio-Rad) for 10 min at 70° C., and magnetized. The eluent was transferred to a new vial before loading SDS-PAGE electrophoresis.

Human pathologic specimens. All human biologic specimens were acquired in accordance with approval from the individual academic medical center institutional review boards (Partners IRB #2016P001640, Duke IRB #Pro00082338, and UW IRB #46425) and informed consent was obtained from patients where applicable. Demographic and clinical data for human pathologic specimens are provided in the Table. Patients with acute pulmonary embolism or deep vein thrombosis were identified at the time of surgical thrombectomy or at autopsy and prepared according to the standard protocol of the Pathology Department at Brigham and Women's Hospital. The affected vasculature was paraffin-embedded, formalin-fixed, and cut into 0.5 m sections on glass slides. Human CTEPH pulmonary endarterectomy specimens were collected as previously described in the manuscript. Proximal and distal segments of chronic thromboemboli were similarly paraffin-embedded, formalin-fixed, and cut into 5 m sections on glass slides for subsequent in vitro analyses.

Histology in vitro. Hematoxylin and eosin (H/E) and Masson's trichrome staining of human pathologic specimens were performed according to methods published previously.³⁸ Briefly, slides were deparaffinized and stained with H/E (Sigma) for histologic analysis. To assess overall collagen deposition, sections were stained with a Masson's Trichrome Staining kit (Fisher Scientific) according to manufacturer's instructions. Fibrosis was analyzed on vessels with an approximate diameter of 20-50 μm, located distal to terminal bronchioles, or in the thromboembolic specimens adherent to the vessel intima, using Fiji (NIH)⁴⁰ and expressed as % collagen by according to the following equation: (collagen signal enhancement/total field signal enhancement)×100.

Immunofluorescence in vitro. Human pathologic specimens were prepared for immunofluorescence as previously discussed. Once deparaffinized, slides were placed in 1× Antigen Retrieval Agent (Boston Bioproducts) and heated in a vegetable steamer (Hamilton Beach) for 20 min to unmask the epitope. The slides were then rinsed in PBS-T, blocked in 10% goat serum (Life Technologies) in PBS for 1 hr at room temperature, and incubated in primary antibody overnight at 4° C. (Table 2). Sections were then incubated with fluorescent secondary antibodies for 1 hr at room temperature before being mounted on glass slides with ProLong® Diamond anti-fade mounting medium with DAPI (ThermoFisher). Images were acquired using a Confocal Laser Scanning Microscope (ZEISS LSM 800 with Airyscan, Jena, Germany), as described previously.³⁹

Control (donor) HPAECs and CTEPH-HPAECs were grown to confluence on chamber slides and fixed with ice cold acetone for 10 min according to methods reported previously.³⁹ The cells were blocked with 10% goat serum (Life Technologies) in PBS for 1 hr at room temperature. Fixed cells were labeled using antibodies against NEDD9, HIF-1α, PECAM-1, or IgG₁ as control (Table 2), or a custom-made monospecific antibody targeting the NEDD9-P1 (msAb-N9-P1) and -P2 (msAb-N9-P2) peptides (see below for methods for generating the mAb-N9s). The secondary antibodies were goat anti-rabbit conjugated with Alexa Fluor 647 and goat anti-mouse conjugated with Alexa Fluor 488 (Abcam). Samples were mounted on glass slides with ProLong® Diamond anti-fade mounting medium with DAPI (ThermoFisher) and imaged using a Confocal Laser Scanning Microscope (ZEISS LSM 800 with Airyscan, Jena, Germany) as described previously.³⁹ Quantitative volumetric analysis was performed on 5 consecutive fields from each sample using the Zen software package algorithm. The Z-stack images were acquired at 0.16 m increments for at least 2.4 m. Fluorescence intensity was quantified using Fiji (NIH).⁴⁰

Animal lung samples were perfused with 10% phosphate-buffered formalin at a pressure of 20 cm H₂O prior to harvesting, were fixed with formalin for at least 24 hr at room temperature, and processed/embedded in paraffin using a Hypercenter XP System and Embedding Center (Shandon, Pittsburgh, Pa.). The paraffin-embedded lung tissue was cut into 5-μm sections and immunofluorescence was performed on sections with distal pulmonary arterioles measuring 20-50 μM in diameter was performed using NEDD9 Ab #1, msAb-N9-P1, msAb-N9-P2, P-Selectin, and IgG₁ (Table 2).

Microscale Thermophoresis. Purified human NEDD9 and P-Selectin/CD62P were purchased from Origene and R&D Systems, respectively. Microscale thermophoresis was performed using a Monolith NT.115pico instrument from NanoTemper Technologies equipped with a pico-RED detector. In these experiments, NEDD9 and P-Selectin served as the target and ligand, respectively. The target was labeled with the RED fluorescent dye NT-647-NHS using Monolith NT™ Protein Labeling Kit RED-NHS (NanoTemper Technologies GmbH, Munich, Germany) according to the manufacturer's instructions. In preliminary studies, we determined that Tris buffer containing 10% glycerol, 1% BSA, 0.05% Tween-20 and 5 mM DDT was an optimal buffer to minimize sample aggregation and adsorption in the capillary tube.³⁹ For experiments, NEDD9 (20 nM) was incubated with decreasing concentrations of P-Selectin (2 μM-0.5 nM) and MST scan was performed. The Hill curves and K_{D} were generated and fit using software from MO.Affinity Analysis v2.2.4 (NanoTemper Technologies, Munchen, Germany).

Isolation of Human Plasma and Platelets. Human blood collection was performed in accordance with the Declaration of Helsinki and ethics regulations with institutional review board approval (Partners IRB #2016P001640). Samples were acquired based largely on availability. Plasma and platelets were isolated from healthy volunteers or patients with CTEPH. Healthy volunteers did not ingest known platelet inhibitors such as aspirin or nonsteroidal anti-inflammatory drugs for at least 10 days prior to blood collection. Venipuncture was performed using a sterile Safety Blood Collection Set+Luer Adapter 21 gauge×¾″ tubing length 12″ (30 cm) (Grenier, #450095) and 10 mL of whole blood was collected in S-Monovette® 10 mL 9NC, Citrate 3.2% (1:10) (Sarstedt, #02.1067.001). For plasma isolation, tubes were spun at 1500×g for 10 min and the plasma was carefully removed with a transfer pipette and stored in 0.5 mL aliquots in Eppendorf tubes at −80° C. until analysis. Platelet isolation was performed according to previously published methods.⁴¹ Briefly, the tubes of whole blood were spun at 177×g for 20 min at room temperature. The platelet-rich plasma (PRP) was collected and 1 μl of diluted PGE₁ (Sigma, P5515-1MG) (1:50 in PBS) was added for every mL of PRP isolated. The PRP was then spun at 100×g for 5 minutes at room temperature and the liquid was aspirated without disturbing the platelet pellet. Platelets were then suspended in wash buffer (140 mM NaCl, 5 mM KCl, 12 mM trisodium citrate, 10 mM glucose, 12.5 mM sucrose, pH 5 6.0) with 1 μl of diluted PGE₁ every mL of PRP, spun again at 100×g for 5 min, incubated with 5-chloromethylfluorescein diacetate (CMFDA) (ThermoFisher) at 1:10,000 dilution for 30 min in a 37° C. water bath, and the wash was repeated for a total of two washes. After the final wash, the platelet pellet was resuspended in platelet buffer (10 mM N-2-hydroxyethylpiperazine-N9-2-ethanesulfonic acid, 140 mM NaCl, 3 mM KCl, 0.5 mM MgCl2, 5 mM NaHCO₃, 10 mM glucose, pH 7.4) and placed in 37° C. water bath for 45 min before treatment.

Activation of Platelets. Platelets were activated in vitro by exposure to 10 μM thrombin receptor-activating peptide (TRAP) (Sigma). Platelets were exposed to agonist for 10 min at 37° C. prior to processing for flow cytometry or incubation with endothelial cells. The activation state of platelets was determined by P-Selectin antibody (Table 2) labeling on flow cytometry (BD Canto II, BD Biosciences).

Platelet Immunofluorescence. Platelet immunofluorescence microscopy was performed according to previously published methods.⁴¹ Rabbit anti-NEDD9, rabbit anti-VEGF antibody, and mouse anti-VEGF antibodies were used (Table 2). Blue phalloidinAlexa 350 was used to probe for actin (Table 2). Resting platelets were fixed for 20 min in a suspension of 8% formaldehyde. Solutions of fixed platelets in suspension were placed in wells of a 24-well microliter plate, each containing a polylysine-coated coverslip, and the plate was centrifuged at 250×g for 5 minutes to attach the cells to the coverslip. Specimens were blocked overnight in phosphate-buffered saline (PBS) with 1% BSA, incubated in primary antibody for 2 hr, washed, and treated with appropriate secondary antibody for 1 hr, and then washed extensively. Preparations were mounted in Aqua polymount from Polysciences (Warrington, Pa.) and analyzed at room temperature using a Confocal Laser Scanning Microscope (ZEISS LSM 800 with Airyscan, Jena, Germany).

Immunogold Electron Microscopy. Samples were prepared according to previously published methods.⁴¹ Briefly, isolated human platelets were fixed with 4% paraformaldehyde in 0.1 M Na phosphate buffer, pH 7.4. After 2 hr of fixation at room temperature, the cell pellets were washed with PBS containing 0.2 M glycine to quench free aldehyde groups from the fixative. Before freezing in liquid nitrogen, cell pellets were infiltrated with 2.3 M sucrose in PBS for 15 min. Frozen samples were sectioned at −120° C., and the sections were transferred to formvar-carbon coated copper grids and floated on PBS until the immunogold labeling performed at room temperature on a piece of parafilm. The rabbit anti-NEDD9 antibody (Table 2) and protein A gold (15 nM) were diluted with 1% BSA. Grids were floated on drops of 1% BSA for 10 min to block for nonspecific labeling, transferred to 5-μL drops of primary antibody, and incubated for 30 min. The grids were then washed in 4 drops of PBS for a total of 15 min, transferred to 5 μL drops of Protein-A gold for 20 min, and washed in 4 drops of PBS for 15 min and 6 drops of double distilled water. Contrasting/embedding of the labeled grids was carried out on ice in 0.3% uranyl acetate in 2% methyl cellulose for 10 min. The grids were examined in a Tecnai G2 Spirit BioTWIN transmission electron microscope (Hillsboro, Oreg.) at 15-25,000× magnification at an accelerating voltage of 80 kV. Images were recorded with an AMT 2k CCD camera.

Liquid Chromatography-Mass Spectrometry. Methods for in-gel trypsin digestion liquid chromatography-mass spectrometry have been reported previously,³⁹ and reiterated here for completeness. Briefly, excised gel bands were cut into approximately 1 mm³ pieces. Gel pieces were then subjected to a modified in-gel trypsin digestion procedure: the gel pieces were washed and dehydrated with acetonitrile for 10 min followed by removal of acetonitrile.^{38,39} Pieces were then completely dried in a speed-vac and dehydration of the gel pieces was achieved with 50 mM ammonium bicarbonate solution containing 12.5 ng/μl modified sequencing-grade trypsin (Promega, Madison, Wis.) at 4° C. After 45 min, the excess trypsin solution was removed and replaced with 50 mM ammonium bicarbonate solution to just cover the gel pieces. Samples were then placed in a 37° C. room overnight. Peptides were later extracted by removing the ammonium bicarbonate solution followed by one wash with a solution containing 50% acetonitrile and 1% formic acid. The extracts were then dried in a speed-vac (˜1 hr) and stored at 4° C. until analysis.

On the day of analysis, the samples were reduced with DTT (Sigma) at a 1 mM concentration (in 50 mM ammonium bicarbonate) for 30 min at 60° C. The samples were then cooled to room temperature and iodoacetamide (stock in 50 mM ammonium bicarbonate) (Sigma) was added to a concentration of 5 mM for 15 min in the dark at room temperature. DTT was then added to a 5 mM concentration to quench the reaction. We then add sequence grade trypsin at a concentration of 5 ng/l. The digestion is over-night at 37° C. The samples are then desalted by an in-house made desalting column. Samples were reconstituted in 5-10 μl of HPLC solvent A (97.5% water, 2.5% acetonitrile and 0.1% formic acid). A nanoscale reverse-phase HPLC capillary column was created by packing 2.6 μm C18 spherical silica beads into a fused silica capillary (100 μm inner diameter×˜30 cm length) with a flame-drawn tip.⁴ After equilibrating the column each sample was loaded via a Famos auto sampler (LC Packings, San Francisco Calif.) onto the column. A gradient was formed and peptides were eluted with increasing concentrations of solvent B (HPLC buffer B=97.5% acetonitrile, 2.5% water and 0.1% formic acid).

As peptides eluted, they were subjected to electrospray ionization and then entered into an LTQ Orbitrap Velos Pro ion-trap mass spectrometer (Thermo Fisher Scientific, Waltham, Mass.). Peptides were detected, isolated, and fragmented to produce a tandem mass spectrum of specific fragment ions for each peptide. Peptide sequences (and hence protein identity) were determined by matching protein databases with the acquired fragmentation pattern by the software program, Sequest (Thermo Fisher Scientific, Waltham, Mass.).⁴³ All databases include a reversed version of all the sequences and the data was filtered to between a one and two percent peptide false discovery rate.

siRNA transfection in vitro. HPAECs were transfected with NEDD9 siRNA (40 nM) or HIF-1α siRNA (20 nM) or scrambled (negative) control siRNAs (Santa Cruz Biotechnology) using Lipofectamine™ 2000 (Invitrogen) for 5 h in OptiMEM® I media, which also served as V control.⁷ The NEDD9 siRNA pool used for transfection was:

Sense (S):
(SEQ ID NO: 12)
5′-GGAUCCUACACCAGUCUAAtt-3′,
Antisense (AS):
(SEQ ID NO: 13)
5′-UUAGACUGGUGUAGGAUCCtt-3′;
S:
(SEQ ID NO: 14)
5′-GAAGGACAUGUGAUCUCAAtt-3′;
AS:
(SEQ ID NO: 15)
5′-UUGAGAUCACAUGUCCUUCtt-3′;
S:
(SEQ ID NO: 16)
5′-GCUAUUGGAGAGCAAUUCAtt-3′,
AS:
(SEQ ID NO: 17)
5′-UGAAUUGCUCUCCAAUAGCtt-3′.

The HIF-1αsiRNA (Dharmacon) pool used for transfection included:

(SEQ ID NO: 18)
5′-GAACAAAUACAUGGGUUA-3′,
(SEQ ID NO: 19)
5′-AGAAUGAAGUGUACCCUAA-3′,
(SEQ ID NO: 20)
5′-GAUGGAACACUAGACAAA-3′,
and
(SEQ ID NO: 21)
5′-CAAGUAGCCUCUUUGACAA-3′.

Exposure to hypoxia. In experiments analyzing the effect of hypoxia on NEDD9 expression, HPAECs were exposed to hypoxia (O₂=0.2%, 2.0% or 10% with N₂ balance at 37° C.) for 24 hr using a modular hypoxia chamber according to methods published previously by our laboratory.^{39,44}

Transgenic NEDD9 mice. Transgenic NEDD9 mice were generously provided by Sachiko Seo at Riken Laboratories. Methods related to the development of these mice were reported previously³⁹ and reiterated here for completeness. A genomic mouse C57BL/6 library was screened with a 300-bp Cas-L probe that included the SH3 region of Cas-L.⁴⁵ A 15-kb clone identified with this probe was subcloned and a targeting vector was constructed using enhanced GFP (pEGFP; Clontech) combined directly with the Cas-L genome at the site of HindIII within exon 2. Incorporation of the vector was accomplished using a neomycin resistance cassette. Electroporation was performed to insert the targeting vector into TT2 embryonic stem cells. Correctly targeted stem cell clones were aggregated with eight cell-stage mouse embryos, and male chimeras were crossed with C57BL/6 females to generate mutant mice. Mice were backcrossed with C57BL/6 mice eight times and bred under pathogen-free conditions. To confirm the correct genotype, genomic DNA from mouse tails was analyzed by Southern Analysis as described and reported previously³⁹ using the following primers: NEDD9 forward: 5′-TCC ACG GTC GCC AAG GCA TTG TCC CAG GGA A-3′ (SEQ ID NO:22); WT reverse: 5′-GCC ATT TAG TAT GTT TGC TTT GGG GC-3 (SEQ ID NO:23)′; NEDD9^{-/-} reverse: 5′-CGG ACT TGA AGA AGT CGT GCT GCT TCA TGT-3′ (SEQ ID NO:24).

Murine Platelet Aggregation Assay. Murine blood collection was performed in accordance with IACUC approval. Wild type and NEDD9^{-/-} mice were anesthetized with a ketamine (80 mg/mL)/xylazine (10 mg/mL) mixture. A heparinized micro-hematocrit capillary tube (Fisher, #22-362-566) was used to extract blood (1,000 μl) from the retro-orbital vein directly into a 1.5 mL Eppendorf tube prefilled with 100 μl of 3.2% citrate until mice were euthanized by exsanguination. The citrated murine whole blood was split into two 500 μl Eppendorf tubes and diluted 1:1 with Hanks' buffer and centrifuged at 177×g for 8 min at room temperature. The PRP was removed in 40 μl aliquots and mixed with 5 μl of different platelet agonists (collagen 0.04-40 μg/mL, PAR4 6.25-200 μM, U46619 0.02-40 μM) in a 1:9 dilution series (in glucose PBS) and negative control (PBS) on 96-well plates and optical density quantified by a plate reader.

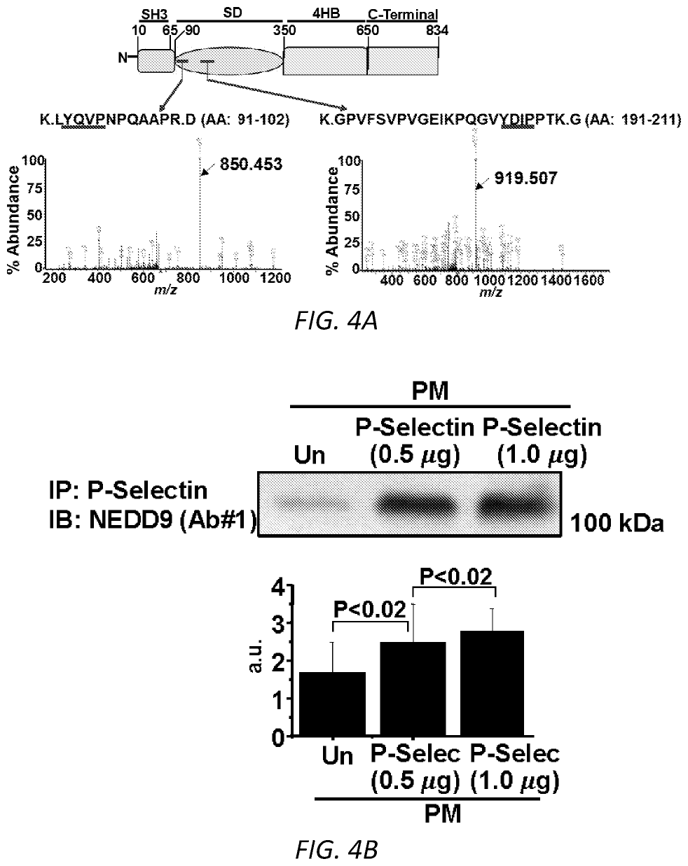

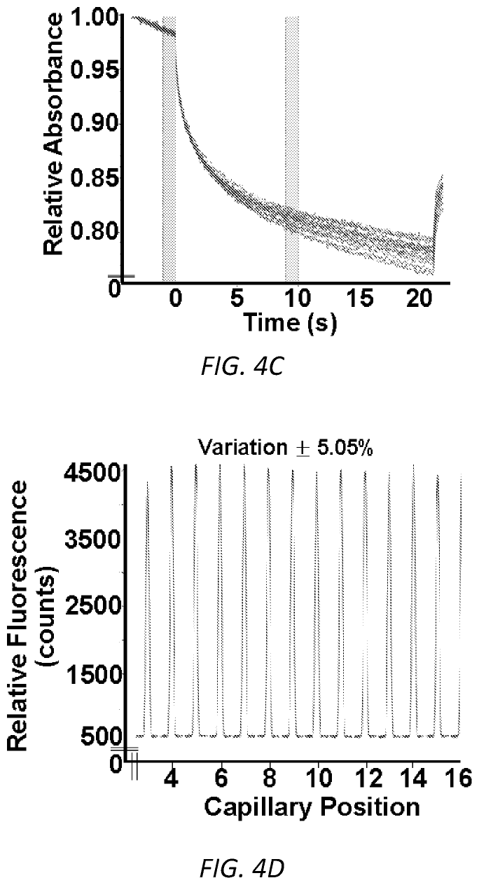

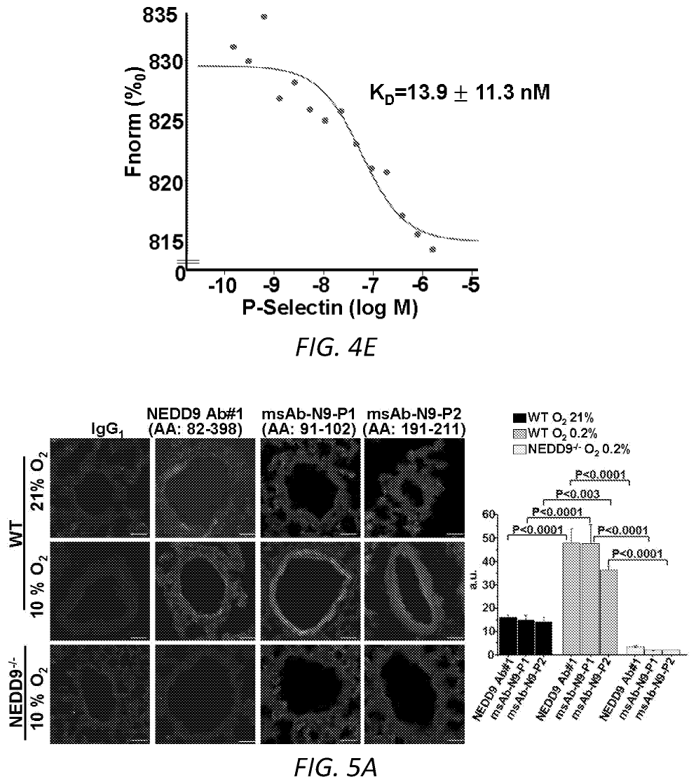

Bleeding Time Assay. Bleeding time was measured through a real-time determination of hemoglobin concentration according to previously published methods.⁴⁶ Briefly, mouse tails were cut and bled into tubes filled with Drabkin reagent (Sigma, D5941) pre-warmed at 37° C. at 15-s intervals. Aliquots were then measured spectrophotometrically at 540 nm. The bleeding time was determined by taking the intersection of the initial slope and the plateau of the plot of hemoglobin concentration versus time, as illustrated in FIG. 4B.

Pulmonary Thromboembolism Model. Mice were anesthetized with a ketamine (80 mg/mL)/xylazine (10 mg/mL) mixture. An incision was made on the ventral side of the neck to expose the right jugular vein. The left jugular vein was exposed, and polythelene-10 tubing (0.011×0.024 inch) (Becton Dickinson) was inserted and secured in the vein. Increasing doses of adenosine diphosphate (ADP) (0.1 to 10 mol/L administered at 80 μL/min at 2-3-min intervals between doses) (Sigma) were administered while the dose-dependent changes in right ventricular systolic pressure (RVSP) to the platelet agonist was monitored.

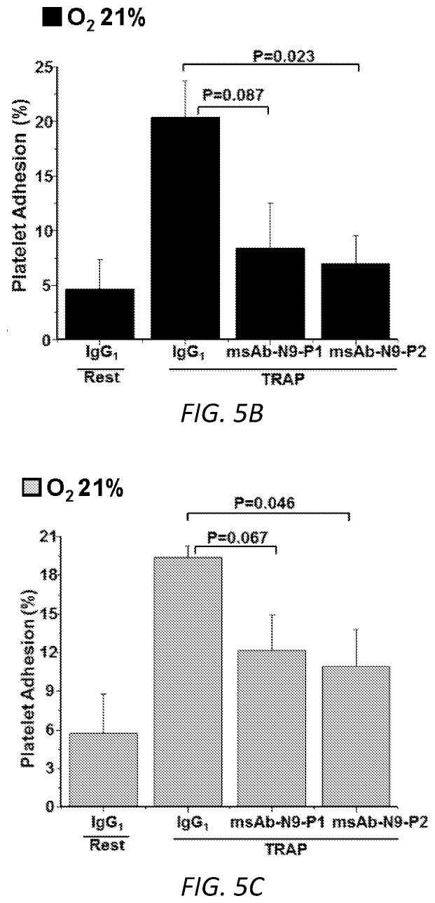

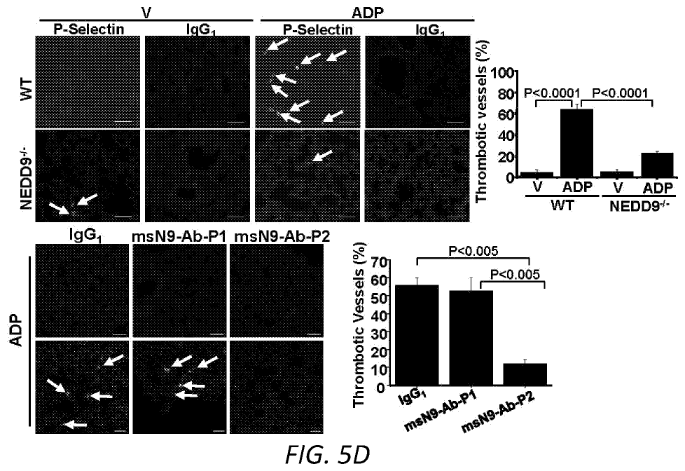

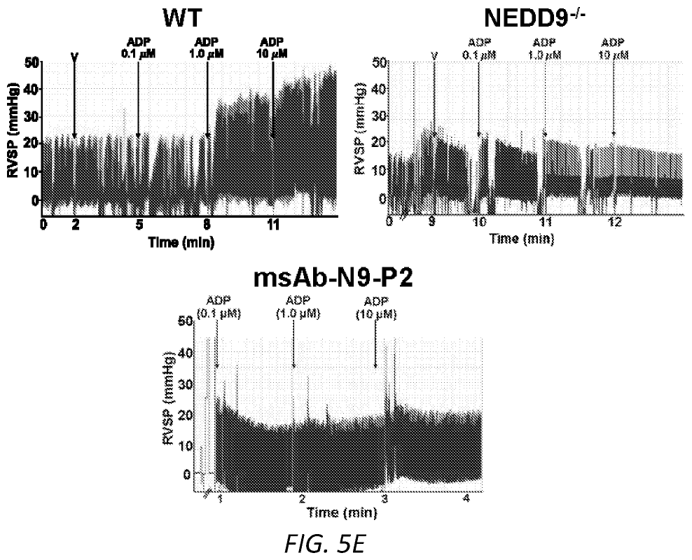

Mouse right heart catheterization. After the left internal jugular was cannulated for ADP infusion as detailed above, the anterior triangle of the right neck was dissected to expose the right internal jugular vein. An Ultra-Miniature Mikro-Tip Pressure Transducer 1.4F catheter (Millar Instruments) inserted into the right jugular vein and advanced into the right ventricle and RVSP was recorded as reported previously.³⁹ All right heart catheterizations were completed within 30 min of sedation induction. RVSP was measured for ˜3 min at steady state with the MPVS 400 System (Millar Instruments). This value was recorded as the baseline RVSP. After each ADP infusion, the new peak RVSP was recorded once a plateau was achieved (˜1 min after infusion). The final peak RVSP was considered the RVSP plateau following the highest ADP dose (10 μM). The difference between final peak RVSP and baseline RVSP was recorded as “A mm Hg from peak to baseline” in FIG. 5E.

Isolation of Plasma Membrane Fractions. HPAECs were grown to 100% confluence in 10-cm cell culture dishes at 37° C., 5.0% CO₂. The plasma membranes were extracted and purified using the Plasma Membrane Protein Extraction Kit (Abcam, #ab65400) according to the manufacturer's instructions. Briefly, the dishes were placed on ice, the culture media was aspirated, and cells were washed with 5 mL ice-cold PBS. Cells were then scraped using a sterile polyethylene cell lifter (Corning Inc.) and centrifuged at 600×g for 5 min at 4° C. The pellet was washed with 3 mL ice-cold PBS before being resuspended in 2 mL of Homogenize Buffer Mix (containing 1:500 dilution of Protease Inhibitor Cocktail) and homogenized using an ice-cold Dounce homogenizer (Sigma). The homogenate was transferred to a 1.5 mL microcentrifuge tube and centrifuged at 700×g for 10 min at 4° C. The supernatant was transferred to a new tube and centrifuged at 10,000×g for 30 min at 4° C. The supernatant (cytosol) was then separated and the pellet (containing proteins from both plasma membrane and cellular organelle membrane) was subsequently purified to isolate plasma membrane proteins specifically. The pellet was resuspended in Upper and Lower Phase Solutions and centrifuged at 3500 rpm (1000×g) for 5 min at 4° C. three times, each time collecting the Upper Phase Solution in a separate tube. The combined Upper Phase Solution was diluted in 5 volumes of distilled H₂O and incubated on ice for 5 min. The solution was then centrifuged at 33,000 rpm for 1.5 hr at 4° C. The pellet (30-50 μg purified plasma membrane protein) was dissolved in 0.5% Triton X-100 in PBS and stored at −80° C. until use.

Monospecific Antibody Preparation. Based on the LC-MS results identifying two NEDD9 substrate domain peptides in the plasma membrane of HPAECs, two immunogenic peptides were synthesized conjugated to keyhole limpet hemocyanin and ovalbumin for immunization and bovine serum albumin for screening. For NEDD9-P1 (aa 91-102), CFGQQKLYQVPNPQAAPRDT-amide (SEQ ID NO:6) was generated, and for NEDD9-P2 (aa 191-211), CGEIKPQGVYDIPPTKGV-amide (SEQ ID NO:8) was generated. Quality control was assured using HPLC analyses. The N9 peptides were slightly modified to increase their immunogenicity, which is why, for instance, 8 AAs (GPVFSVPV; SEQ ID NO:25) from P2 were removed. Two New Zealand white rabbits were then immunized with a mixture of both peptides over 84 days. The rabbits were bled and 5 mL of serum from each reach rabbit was affinity purified over two separate peptide-bound columns to generate purified polyclonal antibodies specific to each peptide.

Plasma NEDD9 Enzyme-Linked Immunosorbent Assay (ELISA). The Aviva Systems Biology NEDD9 ELISA Kit (Human) (OKEH20459) is based on standard sandwich enzyme-linked immunosorbent assay technology and was used according to the manufacturer's instructions. An antibody specific for NEDD9 has been pre-coated onto a 96-well plate. Standards or test samples are added to the wells, incubated, and removed. A biotinylated detector antibody specific for NEDD9 is added, incubated, and then washed. Avidin-peroxidase conjugate is then added, incubated, and unbound conjugate is washed. An enzymatic reaction is produced through the addition of TMB substrate which is catalyzed by HRP generating a blue color product that changes to yellow after adding acidic stop solution. The density of the yellow coloration read by absorbance at 450 nm is quantitatively proportional to the amount of sample NEDD9 captured in the well (ng/mL).

Statistical analyses. Data are expressed as mean S.E.M unless otherwise indicated. For continuous data, comparisons between two groups were performed by the Student's unpaired two-tailed t-test. One-way analysis of variance (ANOVA) was used to examine differences in response to treatments between groups. Post-hoc analysis was performed by the method of Tukey. For categorical variables, the Chi-Square (x²) proportion test was used to examine differences between two groups. The Pearson correlation coefficient is presented for linear regression analyses, which involved normally distributed data. A P<0.05 was considered significant.

TABLE 2
CELL TYPES.

| Cell Type | Source | Lot Number | Sex | Age | Race |
|---|---|---|---|---|---|
| Human Pulmonary Artery | Lonza | 0000598033 | Male | 65 | Caucasian |
| Endothelial Cell | | | | | |
| Human Pulmonary Artery | Lonza | 0000466719 | Male | 57 | Caucasian |
| Endothelial Cell | | | | | |
| Human Pulmonary Artery | Lonza | 4F3041 | Female | 47 | Hispanic |
| Endothelial Cell | | | | | |
| Human Pulmonary Artery | Lonza | 0000657513 | Female | 34 | Caucasian |
| Endothelial Cell | | | | | |
| Human Pulmonary Artery | Lonza | 0000419239 | Male | 34 | Hispanic |
| Smooth Muscle Cell | | | | | |
| Human Coronary Artery | Lonza | 0000662152 | Male | 48 | Caucasian |
| Endothelial Cell | | | | | |
| Human Coronary Artery | Lonza | 18TL036328 | Female | 33 | Caucasian |
| Endothelial Cell | | | | | |
| Human Coronary Artery | Lonza | 0000289727 | Female | 57 | Black |
| Smooth Muscle Cell | | | | | |
| Human Brain Microvascular | Cell | H6023 | * | * | * |
| Endothelial Cells | Biologics | | | | |
| C57BL/6 Mouse Primary | Cell | 092713TZMP | * | * | * |
| Pulmonary Artery | Biologics | | | | |
| Endothelial Cells | | | | | |
| CTEPH Pulmonary | Duke | 3 | Female | 59 | Caucasian |
| Artery Endothelial Cells | University | | | | |
| CTEPH Pulmonary | Duke | 6 | Male | 20 | Caucasian |
| Artery Endothelial Cells | University | | | | |
| CTEPH Pulmonary | Duke | 8 | Male | 35 | Caucasian |
| Artery Endothelial Cells | University | | | | |

* Not provided by Cell Biologics

TABLE 3
BIOLOGICAL REAGENTS.

| | | Lot | |
|---|---|---|---|
| Antibody | Source | Number | Target |
| NEDD9 (Ab | Abeam | ab18056 | Mouse anti-human monoclonal |
| #1) | | (2G9) | antibody targeting fusion protein |
| | | | corresponding to Human |
| | | | HEF1/NEDD-9 AA 82-398 |
| NEDD9 (Ab | Creative | CABT- | Rabbit anti-human polyclonal |
| #2) | Diagnostics | BL2595 | antibody synthetic peptide conjugated |
| | | | to KLH derived from within AA 300- |
| | | | 400 of Human HEF1 |
| NEDD9 (Ab | Abcam | ab110854 | Rabbit anti-human polyclonal |
| #3) | | | antibody targeting a synthetic peptide |
| | | | corresponding to Human |
| | | | HEF1/NEDD-9 (internal sequence) |
| | | | (phospho S369) |
| HIF-1α | Abcam | ab51608 | Rabbit anti-human monoclonal |
| | | | antibody targeting AA 600-700 (C |
| | | | terminal domain) of HIF-1α |
| P-Selectin | Santa Cruz | sc-8419 | Mouse anti-human monoclonal |
| (WB) | | | antibody |
| P-Selectin | Santa Cruz | sc-271267 | Mouse anti-human monoclonal |
| (IP) | | | antibody specific for an epitope |
| | | | mapping between AA 794-829 at the |
| | | | C-terminus of P-Selectin of human |
| | | | origin |
| CD62P/P- | BD | 555524 | Mouse anti-human monoclonal |
| Selectin | Pharmingen | | antibody that targets CD62P, a 140 |
| (FACS) | | | kDa type I transmembrane glycoprotein |
| | | | that is also known as P-Selectin |
| IgG₁ isotype | BD | 555749 | Mouse kappa monoclonal isotype |
| control | Pharmingen | | control |
| (FACS) | | | |
| IgG₁ isotype | Abeam | ab91353 | Mouse kappa monoclonal isotype |
| control (IF) | | | control |
| CD31/ | Novus | NB100- | Rabbit anti-human polyclonal |
| PECAM-1 | Biologics | 2284 | antibody targeting region between AA |
| | | | 700-738 (C-terminus) |
| CD31/ | Santa Cruz | sc-376764 | Mouse anti-human monoclonal |
| PECAM-1 | | | antibody specific for an epitope |
| | | | mapping between AA 699-727 (C-terminus) |
| Na⁺/K⁺ | Abcam | ab185065 | Rabbit anti-human monoclonal |
| ATPase | | | antibody to AA 1-100 |
| | | | (N-terminal domain) |
| Calreticulin | Abcam | ab22683 | Mouse anti-human monoclonal |
| | | | antibody |
| VEGF | Lab Visions | RB-222-R7 | Rabbit anti-human antibody which |
| (Rabbit) | | | recognizes recombinant human VEGF |
| VEGF | Lab Visions | MS-1467- | Mouse anti-human antibody which |
| (Mouse) | | R7 | recognizes 121, 165, and 189 isoforms |
| | | | of human VEGF |
| Alexa Fluor ® | Thermo | A22281 | High-affinity F-actin probe conjugated |
| 350 | Fisher | | to blue fluorescent dye |
| Phalloidin | | | |
| Goat anti- | Abcam | ab150113 | Goat anti-mouse IgG secondary |
| Mouse IgG | | | antibody conjugated with Alexa Fluor |
| secondary | | | 488 |
| antibody | | | |
| Alexa Fluor ® | | | |
| 488 | | | |
| Goat anti- | Abcam | ab150079 | Goat anti-rabbit IgG secondary |
| Rabbit IgG | | | antibody conjugated with Alexa Fluor |
| secondary | | | 647 |
| antibody | | | |
| Alexa Fluor ® | | | |
| 647 | | | |
| Prolong ® | Thermo | P36971 | |
| Diamond | Fisher | | |
| Antifade | | | |
| Mountant | | | |
| with DAPI | | | |
| | | Lot | |
| si-RNA | Source | Number | Target |
| NEDD9/Cas- | Santa Cruz | sc-40794 | Cas-L siRNA is a pool of 3 target- |
| L siRNA | | | specific 20-25 nt siRNAs designed to |
| | | | knock down NEDD9/Cas-L gene |
| | | | expression |
| Control | Santa Cruz | sc-37007 | Control siRNA-A is a non-targeting |
| (scrambled) | | | 20-25 nt siRNA designed as a negative |
| siRNA-A | | | control |
| HIF1α ON- | Dharmacon | L-004018- | A mixture of 4 siRNA provided as a |
| TARGETplus | | 00-0005 | single reagent |
| | | | targeting human HIF-1α |
| Recombinant | | Lot | |
| Protein | Source | Number | Notes |
| NEDD9 | OriGene | TP307200 | |
| P-Selectin | R&D | ADP3-200 | |
| | Systems | | |
| p130Cas | OriGene | TP309133 | Recombinant protein of human breast |
| (BCAR1) | | | cancer anti-estrogen |
| | | | resistance 1 (BCAR1) is synonymous |
| | | | with p130Cas |
| | | Lot | |
| Reagents | Source | Number | Notes |
| Lipofectamine ™ | Invitrogen | 11668019 | |
| Adenosine | Sigma | A2754 | |
| diphosphate | | | |
| (ADP) | | | |
| Drabkin' s | Sigma | D5941 | |
| reagent | | | |
| SureBeads ™ | Bio-Rad | 1614023 | |
| Protein G | | | |
| Magnetic | | | |
| Beads | | | |
| Thrombin | Sigma | T1573 | |
| Receptor | | | |
| Agonist | | | |
| Peptide | | | |
| (TRAP) | | | |
| CellTracker ™ | ThermoFisher | C7025 | |
| Green 5- | Scientific | | |
| chloromethylf | | | |
| luorescein | | | |
| diacetate | | | |
| (CMFDA) | | | |
| Hank's | Invitrogen | 14025076 | |
| Balanced Salt | | | |
| Solution | | | |
| Type II | Worthington | LS004174 | |
| Collagenase | Biochemical | | |
| | Corporation | | |
| Antigen | Boston | BM-745 | |
| Retrieval | Bioproducts | | |
| Agent | | | |
| | | Lot | |
| Assay Kits | Source | Number | Notes |
| Plasma | Abeam | Ab65400 | |
| Membrane | | | |
| Protein | | | |
| Extraction Kit | | | |
| Monolith NT | L001 | Nano | |
| Protein | | Temper | |
| Labeling Kit | | | |
| RED-NHS | | | |
| Masson's | Fisher | 23-900-662 | |
| Trichrome | Scientific | | |
| NEDD9 | Aviva | OKEH204 | |
| ELISA Kit | Systems | 59 | |
| (Human) | Biology | | |

HEF-1 = human enhancer of filamentation -1 (synonymous with NEDD9);
AA = amino acid;
WB = Western blot;
IP = immunoprecipitation;
FACS = fluorescence activated cell sorting;
VEGF = vascular endothelial growth factor;
DAPI = 4′,6-diamidino-2-phenylindole.

### Example 1. Hypoxia Induced HIF-1α-Dependent Upregulation of NEDD9 in HPAECs Selectively

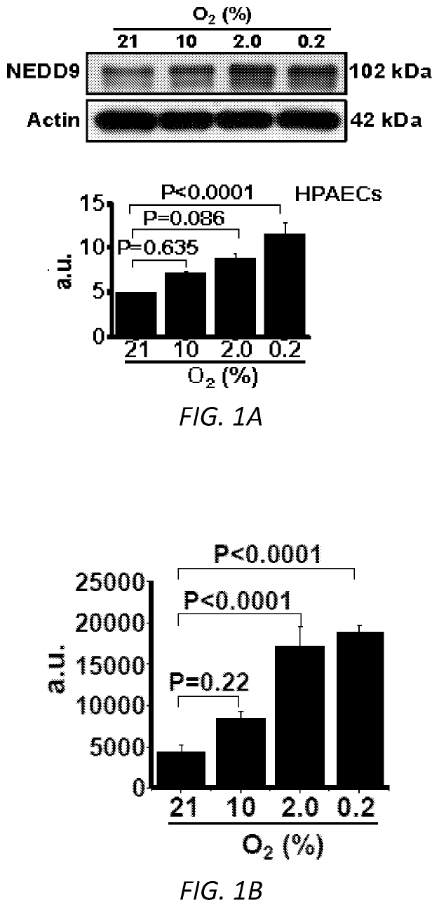

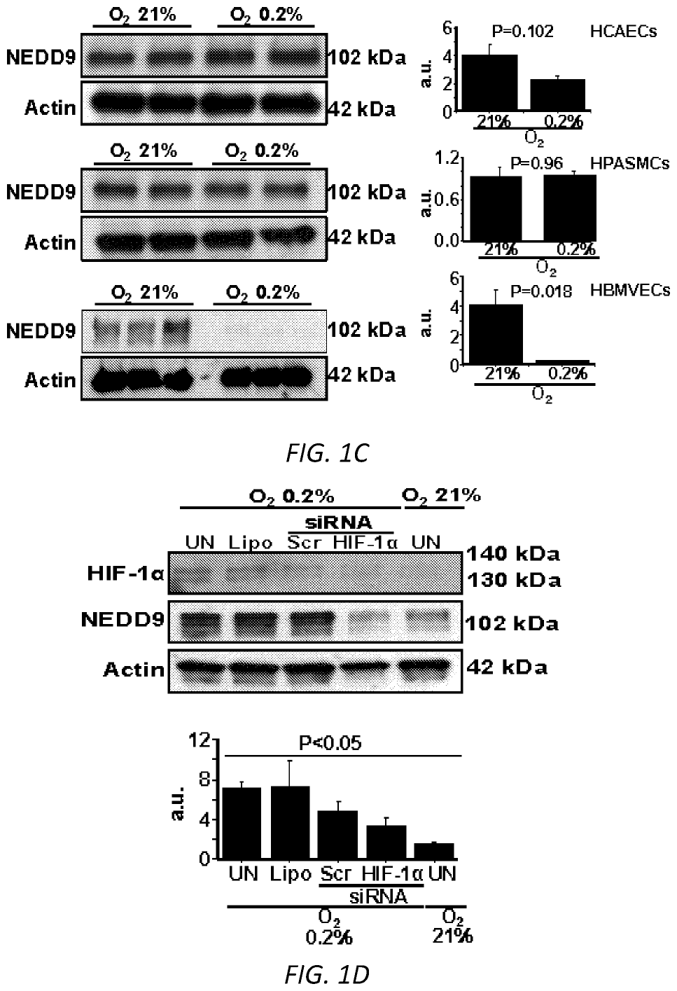

Lysates from cultured HPAECs were treated with normoxia or hypoxia (10%, 2%, and 0.2% O₂) for 24 hr and anti-NEDD9 immunoblot was performed using NEDD9 Ab #1 (Abcam #18056, raised in mouse against human targeting amino acid sequence 82-398).¹¹ Compared to normoxia, hypoxia induced a dose-dependent increase in NEDD9 (4.9±0.1 vs. 6.1±0.2 vs. 7.6±0.6 vs. 12±1.2 a.u., P=0.003 by ANOVA, N=3) (FIG. 1A), which was directionally similar to findings from anti-NEDD9 immunofluorescence (FIG. 1B). However, maximal hypoxia (0.2% O₂) for 24 hr did not affect NEDD9 expression significantly in human pulmonary artery smooth muscle cells (P=0.96, N=3) or systemic vascular cells, including human coronary artery endothelial cells (P=0.102, N=3). In human brain microvascular endothelial cells, the opposite effect was observed, as hypoxia (0.2% O₂) for 24 hr decreased NEDD9 expression significantly compared to normoxia (4.1±1.0 vs. 0.2±0.0 a.u., P=0.018, N=3) (FIG. 1C).

Prior reports in adenocarcinoma cell lines have suggested that hypoxia-NEDD9 signaling is regulated by HIF-1α; thus, we next aimed to determine if a similar mechanism could account for our findings in HPAECs. Compared to cells transfected with vehicle (V)-control (i.e., Lipofectamine™ alone) or scrambled si-RNA (negative) control (si-Scr), transfection of HPAECs with si-HIF-1α for 24 hr decreased NEDD9 expression by 67% and 64%, respectively, and significantly inhibited hypoxia-induced upregulation of NEDD9 by 54% (P<0.05 by ANOVA) (FIG. 1D).

### Example 2. The NEDD9 Substrate Domain Localized to the Extracellular Plasma Membrane of HPAECs

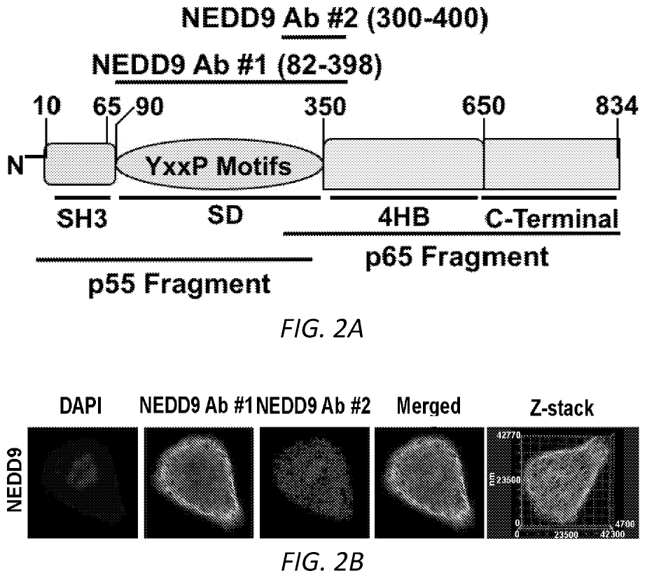

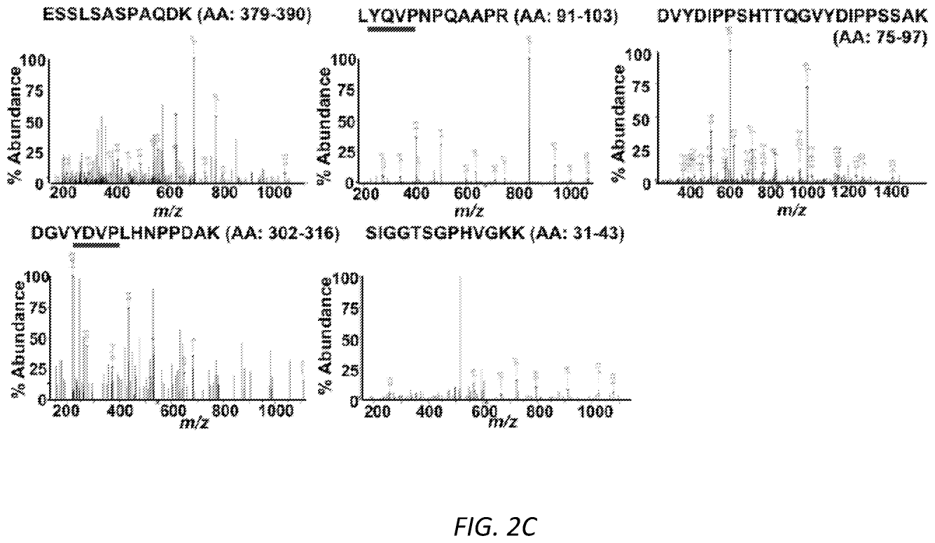

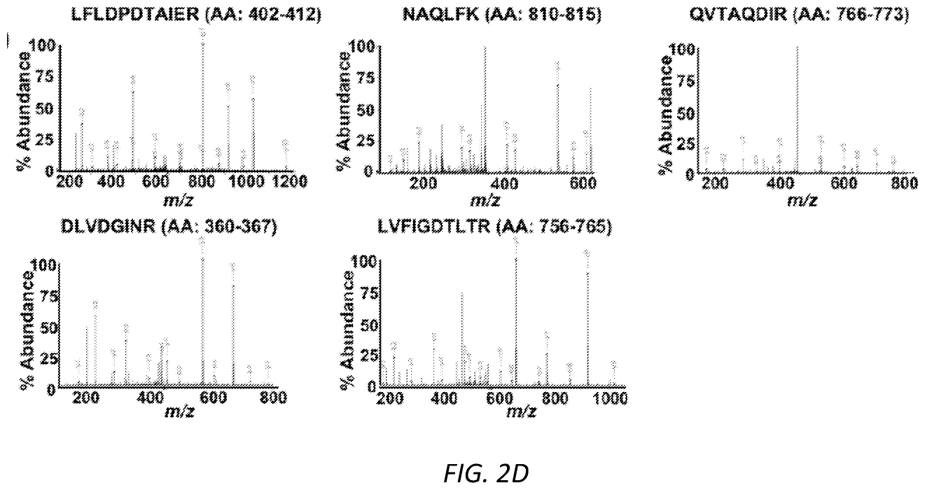

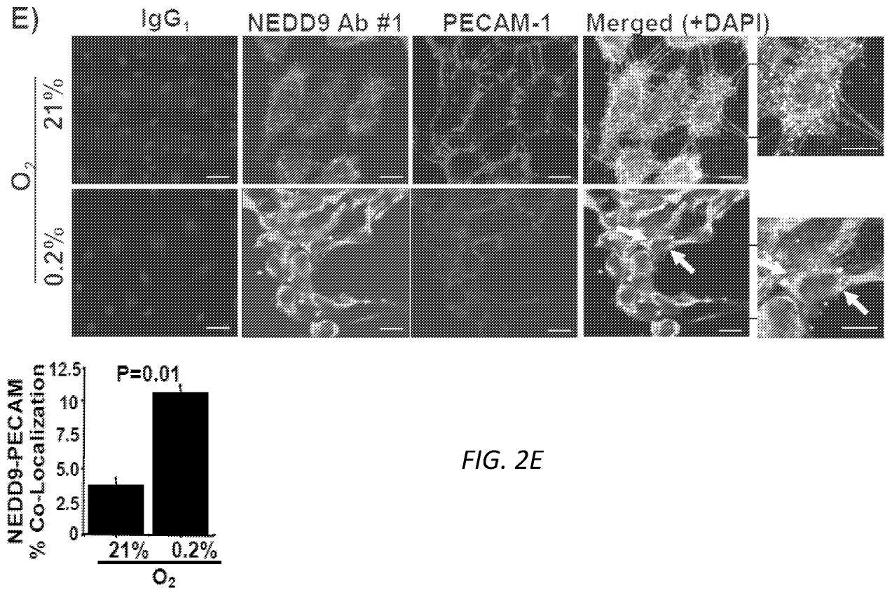

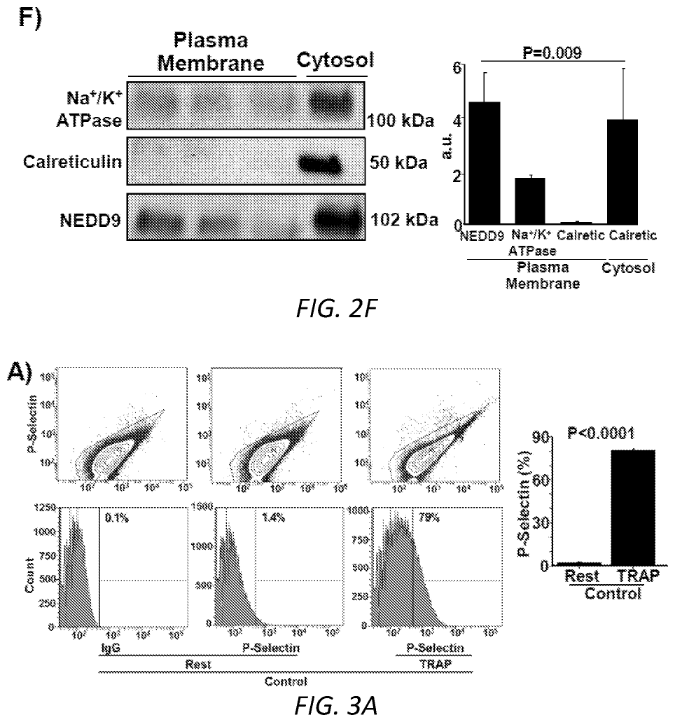

Biologically active protein-protein interactions involving intranuclear and intracytoplasmic NEDD9 have been reported previously.¹¹ However, we hypothesized that NEDD9 regulates platelet-endothelial adhesion directly and, therefore, investigated NEDD9 localization to the plasma membrane. Immunofluorescence demonstrated distinct subcellular expression patterns relative to different NEDD9 antibody targets (FIG. 2A,B). Specifically, NEDD9 was detected at the cell perimeter using NEDD9 Ab #1, which targets the p55 NEDD9 cleavage product, and includes the NEDD9 protein substrate domain (AA: 82-398). By contrast, mainly cytosplasmic NEDD9 was detected using NEDD9 Ab #2 (Creative Diagnostics #BL2595, raised in rabbit against synthetic peptide targeting human amino acid sequence AA: 300-400), which targets a NEDD9 cleavage product (p65 fragment) that includes the protein 4HB and C-terminal domains.^{11,16} This observation was confirmed by Z-stack confocal microscopy (FIG. 2B).

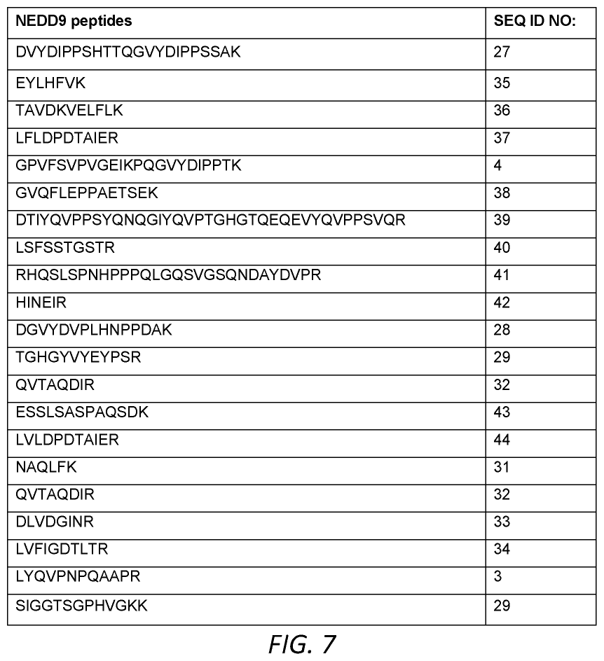

We next performed liquid chromatography-mass spectrometry (LC-MS) on HPAEC lysates immunoprecipitated using anti-NEDD9 Ab #1 and anti-NEDD9 Ab #2 to identify peptides corresponding to each antibody target. We found that NEDD9 Ab #1 bound peptides exclusively in the NEDD9 p55 fragment (FIG. 2C), whereas NEDD9 Ab #2 detected peptides exclusively in the NEDD9 p65 fragment (FIG. 2D) (FIG. 7). Double immunofluorescence demonstrated that compared to normoxia, treatment with hypoxia (0.2% O₂) enhanced colocalization of NEDD9 with the endothelial plasma membrane protein CD31 (PECAM-1) in non-permeabilized HPAECs (3.5±0.7 vs. 11±0.7% co-localization, P=0.01, N=3) (FIG. 2E). These collective data suggested HPAEC plasma membrane expression of the NEDD9 p55 fragment. To support these observations further, we utilized an elution buffer-based methodology. Plasma membrane fractions were isolated and purified from HPAEC lysates, and immunoblot using NEDD9 Ab #1 confirmed NEDD9 expression in the plasma membrane fraction (FIG. 2F).

### Example 3. NEDD9 Modulated Platelet-Endothelial Adhesion In Vitro and Pulmonary Thrombosis In Vivo

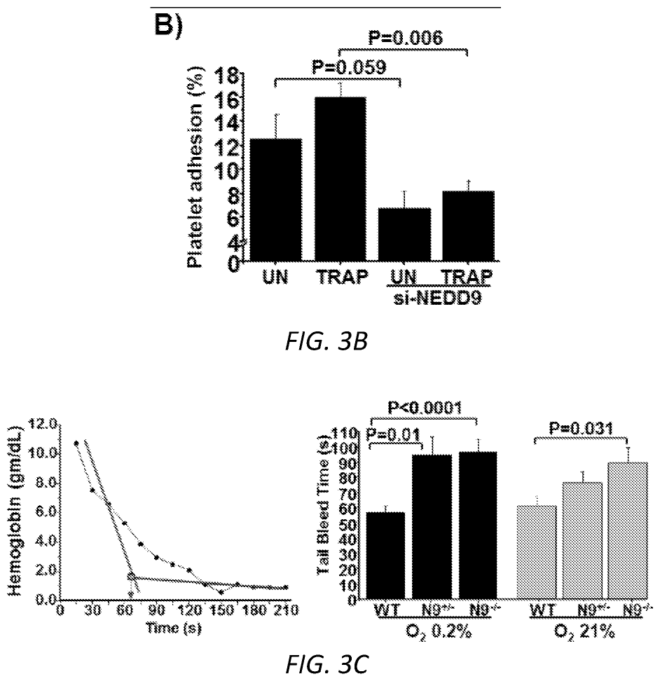

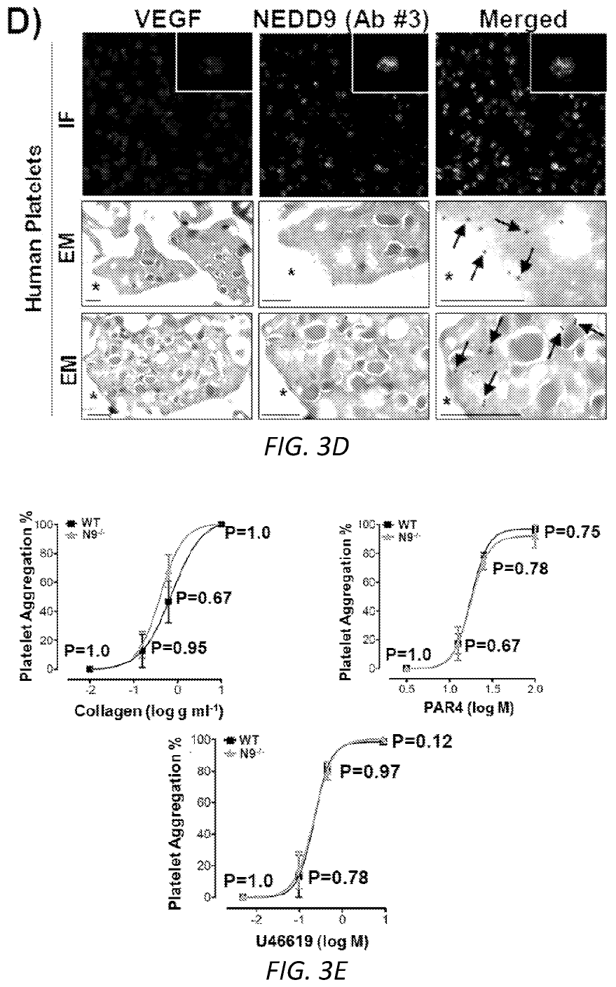

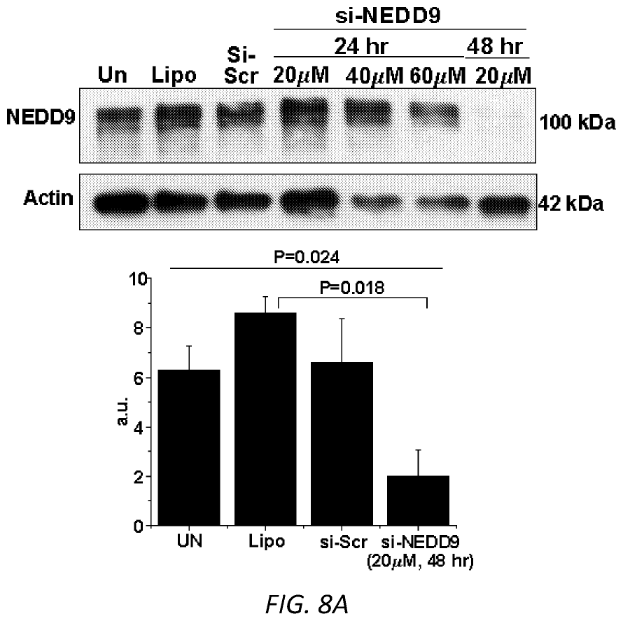

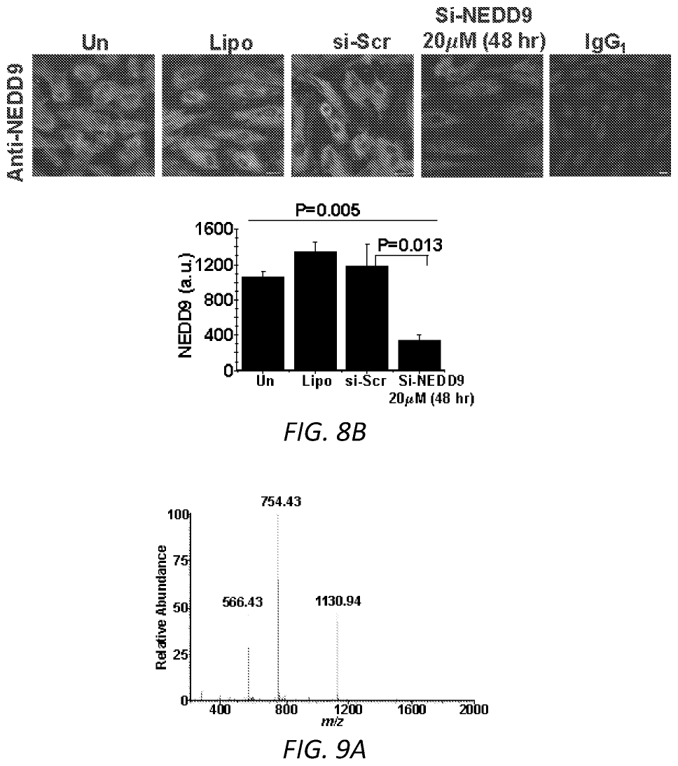

Flow cytometry confirmed activation of platelets by TRAP (10 μM) prior to measuring platelet-HPAEC adhesion assays (FIG. 3A). Compared to untransfected cells, si-NEDD9 (FIG. 8) decreased platelet-HPAEC adhesion under basal conditions (13±4.1 vs. 6.7±2.9% adhesion, P=0.03, N=4) and following platelet stimulation with TRAP (15±0.3 vs. 1.3±0.3% adhesion, P=0.006, N=4) (FIG. 3B). Compared to wild type (WT) mice, the tail bleeding time in transgenic NEDD9-mice was increased significantly under conditions of normoxia (61.1±7.15 vs. 89.7±10.4 s, P=0.031, N=10-12) and after treating mice with hypoxia (10% O₂ for 5 d) (57±2.8 vs. 97±7.9 s, P=0.0001, N=8) (FIG. 3C). Bleeding time has been reported as a measure of intrinsic platelet function,¹⁷ however, raising the possibility that NEDD9 in platelets may affect hemostasis. To explore this further, immunofluorescence and electron microscopy immunocytochemistry using rabbit anti-human NEDD9 Ab #3 (Abcam #110854, raised in rabbit against a synthetic peptide targeting the p55 fragment) was performed on platelets isolated from healthy human controls. We observed NEDD9 expression outside of platelet α-granules by immunofluorescence, and along the outer perimeter of platelets by EM (FIG. 3D). We next isolated platelets from WT and NEDD9^{-/-} mice for platelet aggregometry. However, no difference in global platelet aggregation was observed between WT and NEDD9^{-/-} mice in response to collagen (0.04-40 μg/mL) or other potent murine platelet agonists including protease activator receptor 4 (6.25-200 μM) and 9,11-Dideoxy-9α,11α-methanoepoxy prostaglandin F₂a (U46619) (0.02-40 μM) (FIG. 3E). These data indicate that the principal effect of NEDD9 on bleeding time was due to platelet-endothelial adhesion rather than platelet-platelet aggregation.

### Example 4. P-Selectin Bound the NEDD9 Substrate Domain

Our data suggested that the NEDD9 p55 fragment, which includes the substrate domain, localizes to the HPAEC plasma membrane. The substrate domain is characterized by numerous YxxP motifs, and prior reports have demonstrated that tyrosine is crucial for platelet P-Selectin participation in platelet-endothelial interactions.¹⁸ To determine if P-Selectin may target the NEDD9 substrate domain in HPAECs, plasma membrane fractions incubated with recombinant P-Selectin for 1 hr were immunoprecipitated with an anti-P-Selectin antibody. Next, LC-MS was performed on in-gel trypsin-digested lysates, and identified only two NEDD9 peptide sequences, both within the substrate domain: K.LYQVPNPQAAPR.D (AA: 91-102; SEQ ID NO:9) (N9-P1) and K.GPVFSVPVGEIKPQGVYDIPPTK.G (AA: 191-211; SEQ ID NO:10) (N9-P2) (N=2 replicates for N=2 iterations) (FIG. 4A). The “.” at either end of these sequences indicate that this was the cleavage site of the peptide, and, therefore, the target amino acids are between the “.” symbols. Plasma membrane fractions from HPAECs were incubated with V-control or exogenous (recombinant) P-Selectin for 1 hr, and P-Selectin-NEDD9 complex formation was assessed by co-immunoprecipitation. Compared with V-control, P-Selectin (1.0 μg) increased NEDD9:P-Selectin complex formation significantly by 3-fold (P=0.02, N=3) (FIG. 4B).

The formation of a P-Selectin-NEDD9 complex has important implications on thrombosis, but has not been reported previously. Therefore, we next used microscale thermophoresis, which detects temperature-induced changes in fluorescence of a target to quantify high-affinity biomolecular interactions.^{11,19} Varying concentrations of P-Selectin (ligand) (2 μM-0.5 nM) were co-incubated with NEDD9 (receptor) (20 nM), which resulted in a dose titration curve profile indicative of definitive physical association between the receptor and ligand (N=2) (FIG. 4C-E).

### Example 5. NEDD9 is a Modifiable Target to Inhibit Platelet-Endothelial Adhesion In Vitro

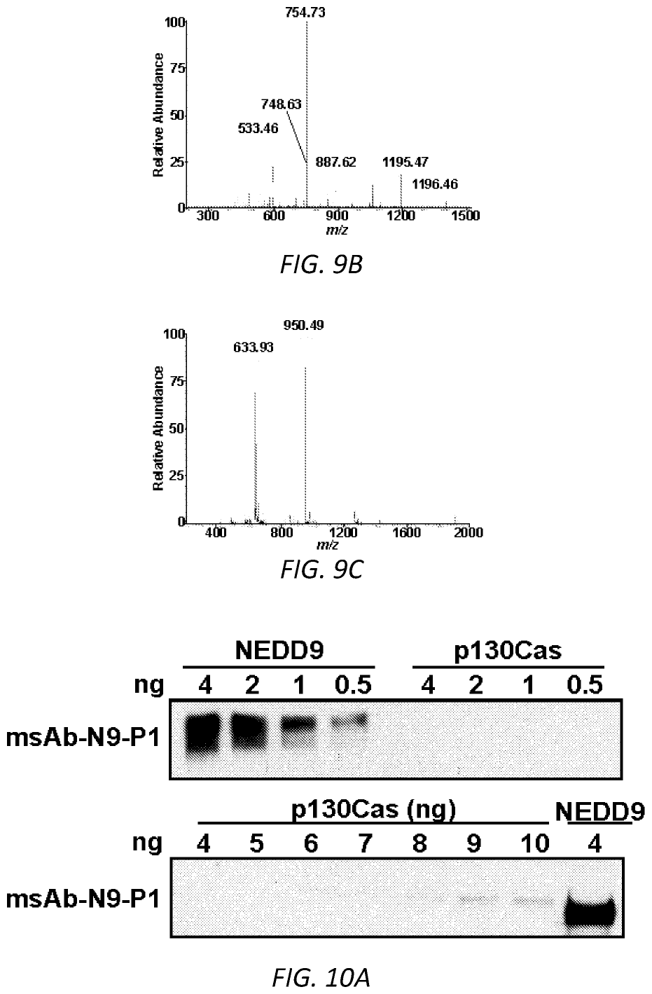

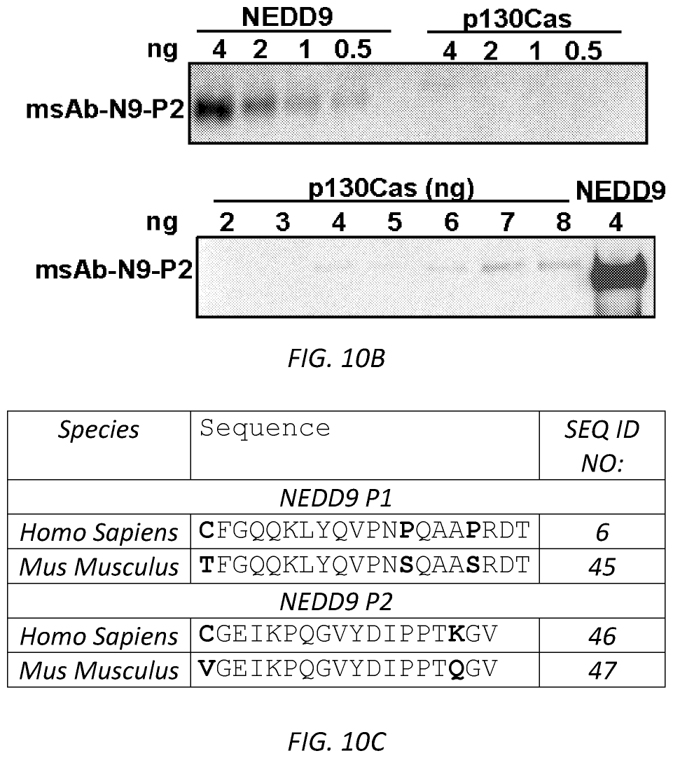

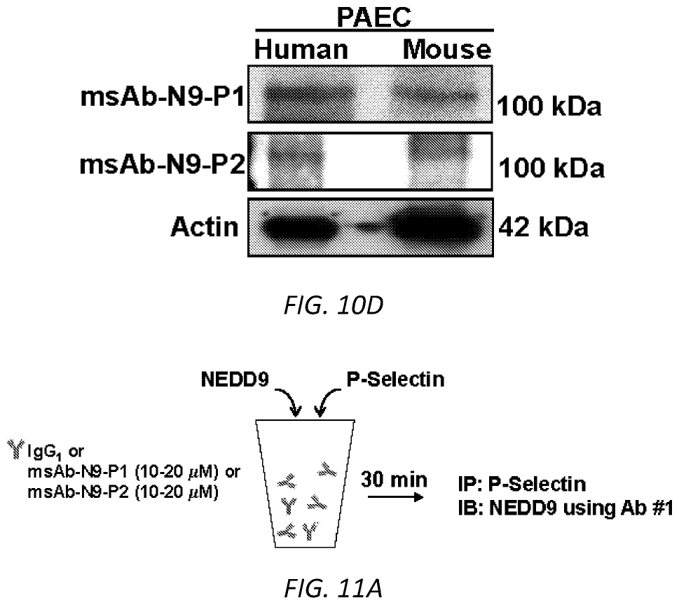

We next aimed to determine if N9-P1 or N9-P2 are potential therapeutic targets by which to inhibit platelet-endothelial adhesion. To accomplish this end, two model peptides corresponding to the N9-P1 and N9-P2 sequences were synthesized, and the sequence was confirmed by LC-MS (FIG. 9A-C). These peptides were injected into separate New Zealand white rabbits and used to develop an anti-human, monospecific polyclonal antibody against each peptide (msAb-N9-P1 and msAb-N9-P2). The lower detection threshold for recombinant NEDD9 by msN9-Ab-P1 or msN9-Ab-P2 was 0.5 ng (P<0.05, N=3) by immunoblot. Cross-reactivity for either msAb-N9-P1 or msAb-N9-P2 with p130Cas (≤0.5 ng), which shares 75% amino acid similarity with NEDD9, was, however, not observed (P=1.0, N=3) (FIG. 10A,B).

There is high homology for the amino acid sequence of N9-P1 and N9-P2 across human and murine species (FIG. 9C), and no meaningful difference was observed in NEDD9 detection by msAb-N9-P1, or msAb-N9-P2 between HPAECS and PAECs from control mice (FIG. 10D). Compared to IgG₁ control, anti-msAb-N9-P1 or anti-msAb-N9-P2 immunofluorescence performed on paraffin-embedded pulmonary arterioles (measuring 10-15 μm in diameter) confirmed expression of NEDD9 P1 and P2 in WT C57BL/6 background mice. In turn, there was no difference in NEDD9 detection by msAb-N9-P1 or msAb-N9-P2 compared with IgG₁ control in transgenic NEDD9^{-/-} mice (208±12.6 vs. 187±10.0 vs. 192±0.07 a.u., P=0.36, N=3) (FIG. 5A).

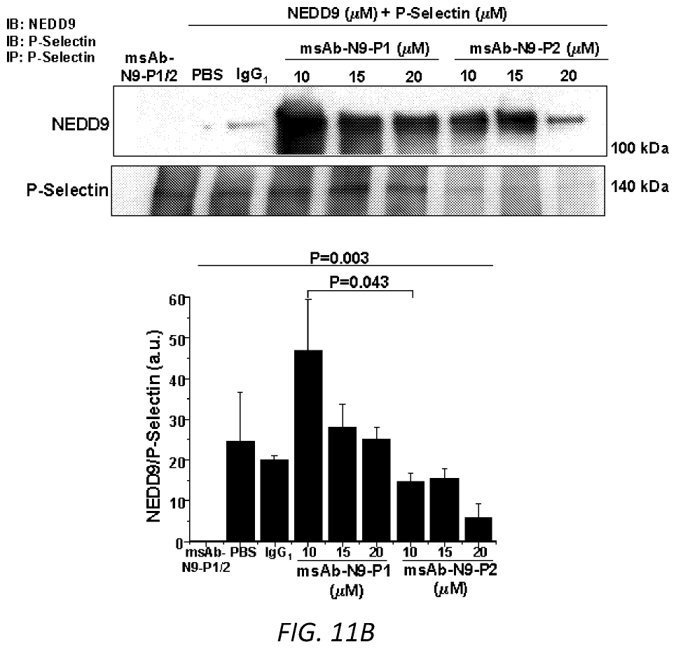

These findings suggested that our custom-made antibodies were specific to NEDD9 with suitable NEDD9 detection across species. Therefore, we next explored the functional effects of msAb-N9-P1 and msAb-N9-P2 on platelet-endothelial biology. First, recombinant NEDD9 and P-Selectin were incubated for 30 min in a cell-free system supplemented with either msAb-N9-P1 (10-20 μM) or msAb-N9-P2 (10-20 μM), and differences in NEDD9-P-Selectin complex formation were analyzed by immunoprecipitation-immunoblot assay. We observed a dose-dependent decrease in NEDD9-P-Selectin complex formation by treatment with msAb-N9-P1 and msAb-N9-P2 (P=0.003 by ANOVA, N=3) (FIG. 11). Co-incubation of normoxia-treated HPAECs with msAb-N9-P1 and msAb-N9-P2 also significantly inhibited TRAP-stimulated platelet-endothelial adhesion (4.6±1.7 [msAb-N9-P1+TRAP] and 4.4±1.3 [msAb-N9-P2+TRAP] vs. 27±9.6 [TRAP alone] % adhesion respectively, P=0.04 by ANOVA, N=3) (FIG. 5B), but inhibition of TRAP-stimulated platelet-endothelial adhesion was mediated only by msAb-N9-P2 in hypoxia-treated cells (19±3.0 vs. 11±2.8% adhesion, P=0.046, N=3) (FIG. 5C). Based on these results, we focused on the effect of msAb-N9-P2 in further experiments involving pulmonary thrombosis in vivo.

### Example 6. NEDD9 Inhibition Prevented Pulmonary Thrombosis and Pulmonary Hypertension In Vivo

To explore the translational relevance of our in vitro findings, we turned to the established murine model of adenosine diphosphate (ADP)-induced pulmonary thrombosis and pulmonary hypertension.²⁰ This experimental model was selected because ADP is a potent stimulator of platelet-endothelial adhesion, which is a critical initial step in the pathogenesis of CTEPH,²¹ and validated experimental models that do not hinge on genetic coagulopathies²² or mechanical trauma²³ to recapitulate the CTEPH vasculopathy are lacking. Compared to WT mice, NEDD9^{-/-} mice were resistant to ADP-induced pulmonary arteriolar thrombotic occlusion analyzed by anti-P-Selectin immunofluorescence (65±2.0 vs. 23±1.8%, P<0.01, N=3/condition) (FIG. 5D), and had a blunted increase in right ventricular systolic pressure (RVSP) (31±9.5 vs. 2.4±2.3 mmHg A from baseline, P=0.05, N=4/condition) (FIG. 5E, Table 4).

To determine if NEDD9 antagonism affects platelet-endothelial adhesion in vivo, WT mice were pre-treated with msAb-N9-P2 for 10 min prior to ADP infusion. Compared to IgG₁ control, treatment with msAb-N9-P2 decreased ADP-induced pulmonary arteriolar thrombotic occlusion (56±7.3 vs. 12±6.1% occlusion, P<0.001, N=3) and pulmonary hypertension (14±3.5 vs. 2.2±0.5 RVSP mmHg A from baseline, P=0.003, N=6) (FIG. 5D,E; Table 4) to levels consistent with our findings in NEDD9^{-/-} transgenic mice.

TABLE 4

| | | | RVSP (mmHg) | | |
|---|---|---|---|---|---|
| | Weight | HR | | | ΔPeak- |
| Condition | (g) | (bpm) | Baseline | Peak | Baseline |
| WT | 25 ± 1.0 | 288 ± 21 | 23 ± 2.4 | 45 ± 1.8* | +22 ± 4.2 |
| NEDD9_{-/-} | 23 ± 0.6 | 280 ± 26 | 27 ± 1.3 | 29 ± 1.0 | +2.3 ± 2.3** |
| WT IgG | 23 ± 0.5 | 308 ± 39 | 16 ± 2.1 | 30 ± 5.4# | +14 ± 3.5 |
| WT msAb-N9- | 24 ± 0.7 | 285 ± 19 | 22 ± 4.0 | 36 ± 3.1 | +14 ± 3.1 |
| P1 | | | | | |
| WT msAb-N9- | 23 ± 0.6 | 330 ± 36 | 18 ± 2.2 | 20 ± 2.5 | +2.2 ± 0.5** |
| P2 | | | | | |

HR, heart rate in beats per minute; RVSP, right ventricular systolic pressure.

### Example 7. NEDD9 is Increased in CTEPH

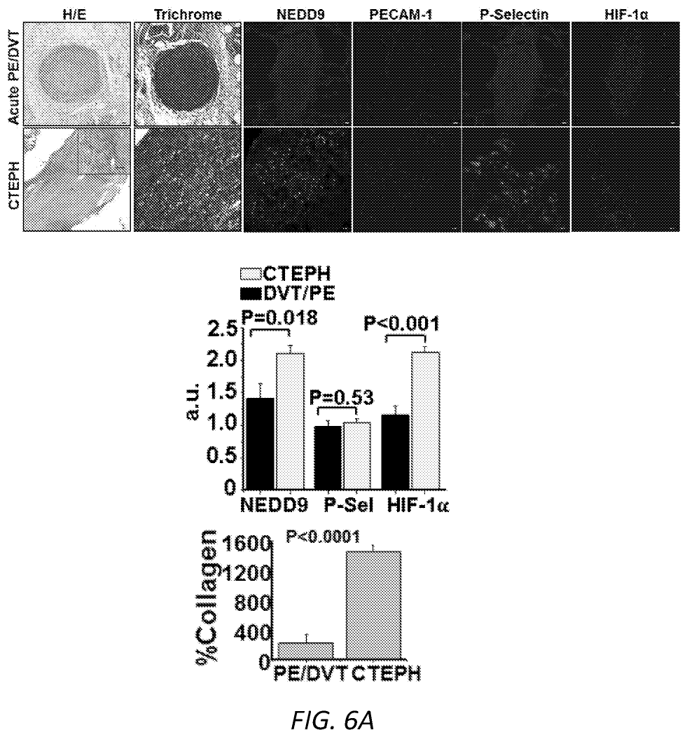

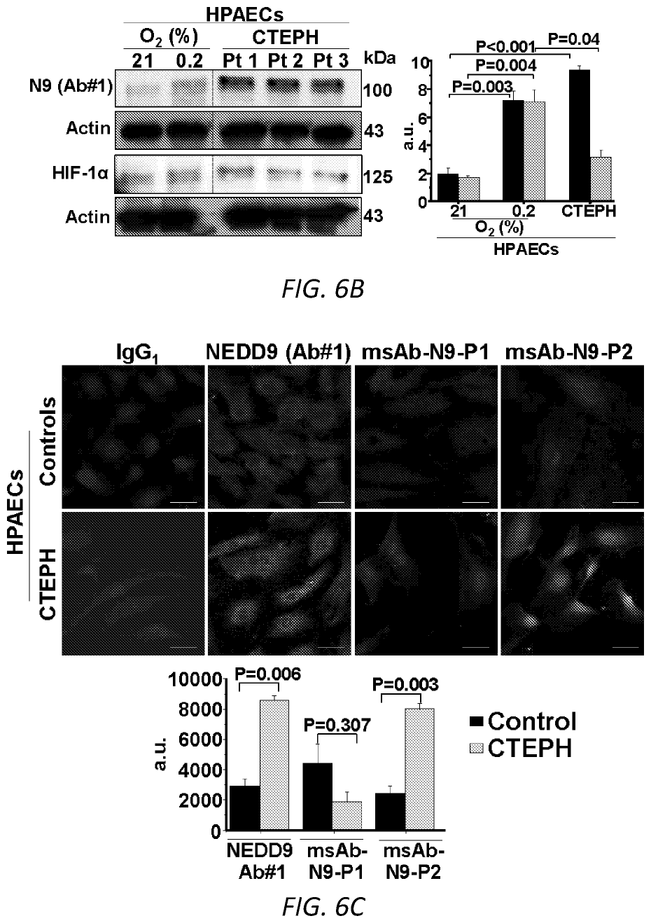

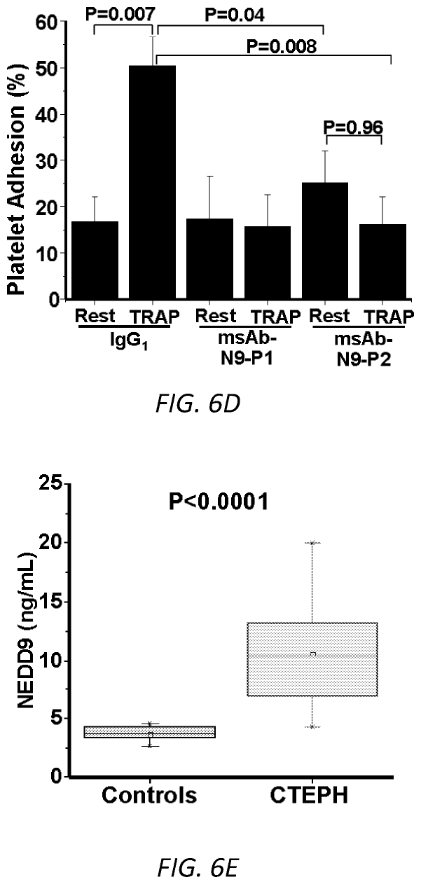

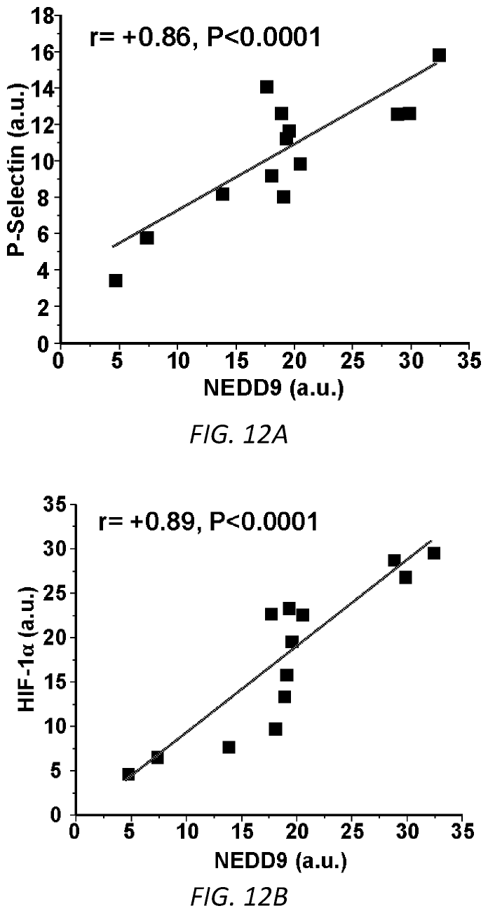

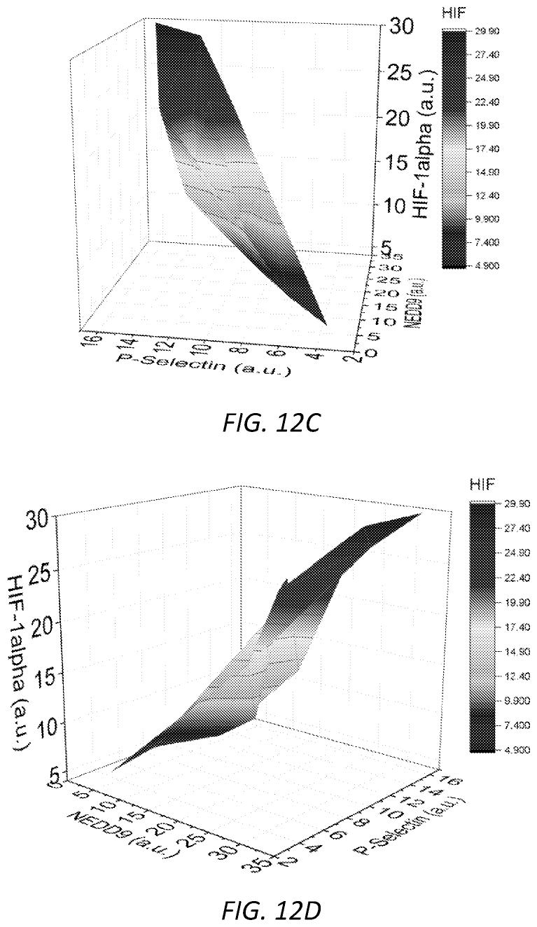

Compared to acute pulmonary embolism and deep vein thrombosis (PE/DVT) (N=6) specimens (disease controls), CTEPH-PEA specimens (N=7) were highly fibrotic (225±163 vs. 1450±94.8% collagen, P<0.0001) and characterized by organizing thrombus and intimal hyperplasia with segments of attached tunica media (FIG. 6A). Immunofluorescence analyses showed that CTEPH-PEA was also characterized by increased expression of HIF-1α (2444±435.6 vs. 7525±530.4, P<0.0001, N=7) and NEDD9 (3223±1293 vs. 7280±730, P<0.0001, N=7), as well as P-Selectin-NEDD9 co-localization in platelet aggregates (2.8±0.3 vs. 10±0.5, P<0.0001, N=7) compared with DVT/PE controls (FIG. 6A). Analyzing the DVT/PE and CTEPH-PEA specimens collectively, NEDD9 correlated strongly with P-Selectin (r=+0.86, P=0.004) and HIF-1α (r=+0.89, P=0.04) (FIG. 12A,B), and the relationship between all three of these variables is shown using an xyz matrix plot in FIG. 12C,D.

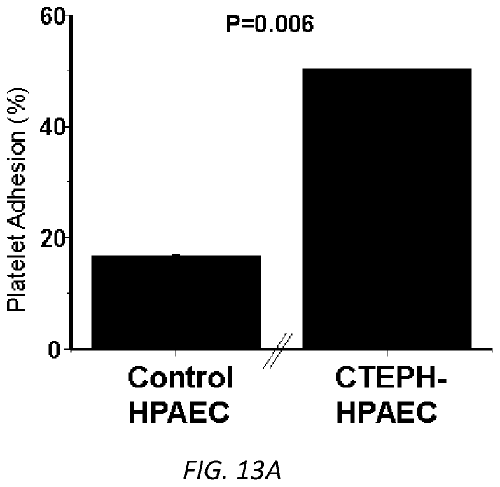

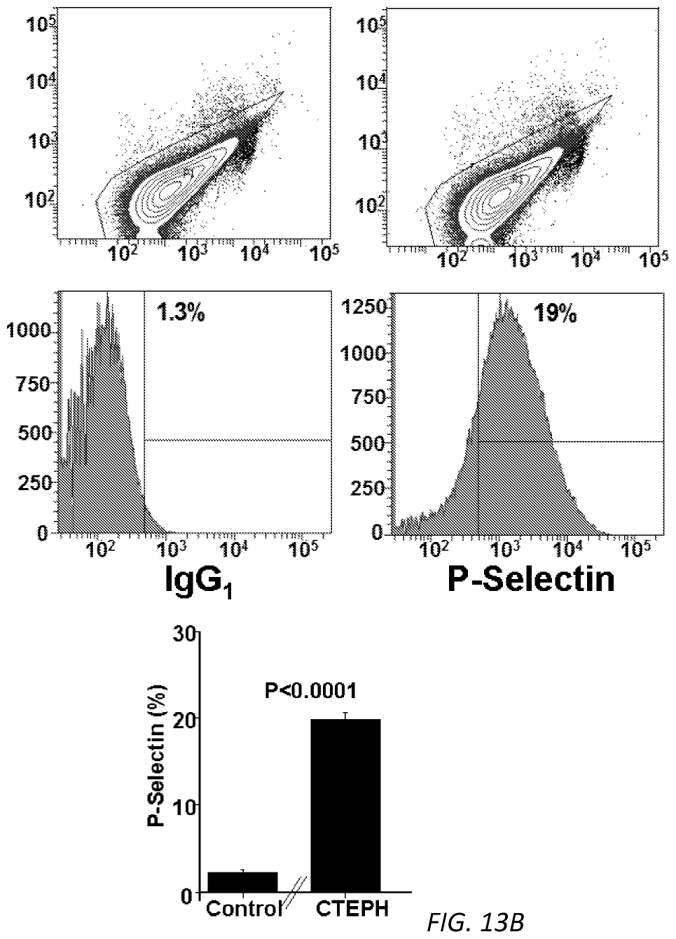

These data were consistent with our findings in HPAECs isolated from CTEPH patients, which expressed increased HIF-1α (1.6±0.2 vs. 3±0.4 a.u., P=0.04, N=3) and NEDD9 (1.98±0.5 vs. 9.3±0.7 a.u., P<0.001, N=3) by immunoblot compared to control HPAECs (FIG. 6B). Immunofluorescence analyses of CTEPH-HPAECs also demonstrated increased NEDD9 expression using NEDD9 Ab #1 and msAb-N9-P2; however, similar findings were not observed by msAb-N9-P1 (FIG. 6C). Akin to prior reports indicating that CTEPH is associated with a prothrombotic endothelium,²⁴ platelet-endothelial adhesion was increased in CTEPH-HPAECs compared with control HPAECs under basal conditions as well as following stimulation of healthy donor platelets with TRAP (16.8±0.05 vs. 50.3±0.06% adhesion, P=0.006, N=4) (FIG. 13A). Despite enhanced thrombogenicity in CTEPH-HPAECs, msAb-N9-P2 inhibited TRAP-stimulated platelet adhesion significantly (50.3±0.06 vs. 16.2±0.06 vs. % adhesion, P=0.007, N=4) (FIG. 6D). In plasma from CTEPH patients, increased platelet activation was observed compared to controls (N=3) (FIG. 13B), as well as increased NEDD9 levels (N=27) compared with age- and sex-matched healthy volunteers (N=7) (9.8±0.7 vs. 3.7±0.2, P=0.0001) (FIG. 6E).

### Example 8. Generating Monoclonal Antibodies to NEDD9

The development of a mAb-N9 is accomplished as follows. Briefly, an immunogenic boost using the NEDD9-P1 and -P2 peptide is administered to the same rabbit(s) used to generate the pAb-N9 (currently age 8 mo., total immunogenic lifespan ˜2 years). Following the rabbit bleed, the NEDD9 titer of unpurified sera is performed by ELISA and as follows: NEDD9-P1 and -P2 (1-5 ng) are loaded on an SDS-PAGE gel. Protein is transferred to a PVDF membrane, which is then incubated with the rabbit sera (5 serial dilutions). The NEDD9 peptide target (e.g. NEDD9-P1 or -P2) demonstrating the highest NEDD9 detection yield is prioritized for use in further experiments. Next, the rabbit spleen is removed, frozen, and analyzed for isolation of B-cells that secrete the preferred NEDD9 peptide target, and a random target as vehicle control (vAb). Next, the variable heavy and variable light genes are isolated and used to generate 5 mAb-N9 clones for testing.

Selection of mAb-N9 clone is performed as follows. All mAb-N9 clones and vAb are analyzed for NEDD9 specificity (vs. p130Cas as performed in FIG. 10A,B) by immunoblot using recombinant human NEDD9. Similarly, mAb-N9 and vAb cross-reactivity analysis with mouse NEDD9 and rat NEDD9 is performed by immunoblot in cultured PAECs and IF in paraffin-embedded lung tissue sections co-stained with an anti-PECAM antibody to localize endothelial signal, respectively. The mAb-N9 clone(s) demonstrating optimal specificity and species cross-reactivity is selected for maxi-prep using the corresponding mAb sequence or plasmid.

### Example 9. NEDD9 in Human Disease and Animal Models

To show that mAb-N9 is effective in human disease samples, we study the effect of mAb-N9 and vAb on platelet-endothelial adhesion using PAECs from normal human volunteers, CTEPH patients, and PAH patients. Success is defined as a reduction in platelet-HPAEC adhesion in CTEPH or PAH by mAb-N9 to within 20% of controls.

Complete dose-finding and tissue distribution experiments are performed to test mAb-N9 in vivo. A series of dose-finding and plasma half-life experiments is performed in which mAb-N9 (0.1-1.0 mg/kg) or vAb in PBS is administered to untreated mice and rats or mice and rats exposed to hypoxia (0.2% O₂) for 3 days to increase PAEC plasma membrane NEDD9 expression. After protocol completion, lungs are cut in cross-section, formalin fixed, and embedded in paraffin. Next, anti-mAb-N9 co-localization with anti-PECAM by IF is completed, visualized by confocal microscopy (Zen), and quantified using ImageJ (NIH). In addition, vascular endothelial membrane NEDD9 levels are quantified in brain, liver, spleen, colon and renal arteries and expressed relative to PAEC membrane NEDD9 expression. In addition, the following experiments are performed.

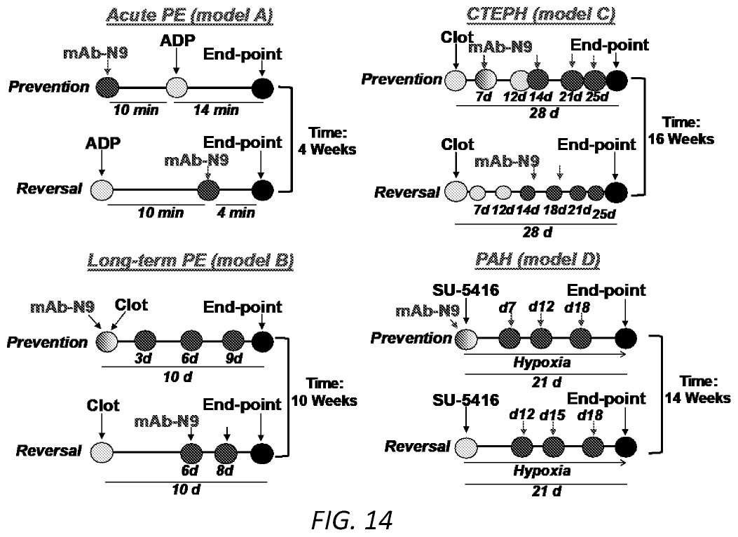

PVTE treatment with mAb-N9: A summary of the PVTE animal models, treatment time points, and expected time to complete experiments is provided in FIG. 14. All animals will be randomized in a 1:1 ratio to receive mAb-N9 (dose and mAb-N9-P1 vs. -P2 selection per results in Aim 3.1) or vAb as control. Data acquisition/analysis will be performed by a PE/PAH expert blinded to treatment condition.

Acute PE: ADP-induced pulmonary embolism. In this murine model, acute activation of platelets with ADP administered by right heart catheterization is leveraged to induce acute PE. The primary end-points used to determine success in this model will be thrombus burden quantified by measuring anti-P-selectin IF detected in pulmonary arterioles and change in right ventricular systolic pressure (RVSP) following ADP administration (see FIGS. 5D-E).

Long-term assessment of luminal PE. In this model, orbital vein blood is collected from anesthetized Sprague Dawley rats and placed in a tissue culture dish for 18 hr. Next, blood clots are washed with normal saline, cut to 3 mm in length, and then injected into the left jugular vein (Deng et al. Sci Rep 2017; 7:2270). In the long-term luminal PE condition, 3 clots are administered at time point 0, and rats will be analyzed at protocol day 10 by echocardiography to assess RV systolic function (tricuspid annular plane of systolic excursion (TAPSE)), right heart catheterization to assess RVSP and other cardiopulmonary hemodynamics including pulmonary vascular resistance (PVR) and cardiac output (CO), and thrombus burden quantified by measuring anti-P-selectin IF detected in pulmonary arterioles. The primary end-points used to determine success in this model will be thrombus burden, TAPSE, RVSP, PVR and CO.

CTEPH To recapitulate CTEPH experimentally, the long-term luminal PE model is used as indicated above in (B), but repeat injection of autologous clot will be administered on protocol days 7 and 12, and hemodynamic/histological assessment will be analyzed on protocol day 28. In addition to thrombus burden, cardiopulmonary hemodynamics and TAPSE, volumetric analysis of pulmonary arterial pruning and tapering assessed by high resolution, contrast enhanced thoracic computed tomography will also be analyzed as a primary end-point (Satoh et al. Circ Res 2017; 120:1246-62).

PVTE in PAH: Sugen-5416-hypoxia-PAH. Sprague-Dawley rats (˜225 g) will be administered a single subcutaneous injection of the VEGFR-2 kinase inhibitor Sugen-5416 (20 mg/kg; Sigma), exposed immediately to chronic hypoxia (10% O₂) until completion of the protocol 21 days later (Samokhin et al. Sci Transl Med. 2018; 10:445). This established PAH model is associated with occlusive thrombotic remodeling of pulmonary arterioles and severe pulmonary hypertension. Primary end-points in this model will be: thrombus burden, RVSP, PVR, and CO.

Secondary end-points for all models: prothrombin time (PT) (Fisher), partial thromboplastin time (aPTT) (Fisher), factor Xa level (Millpore), platelet count (Battinelli Lab) and hemoglobin (Sigma-Aldrich), which provide serological/biochemical data on coagulation, hemostasis, and bleeding, respectively.

TABLE 5

| | Acute PE | Chronic PE | CTEPH | PAH |
|---|---|---|---|---|
| 1° End-points | | | | |
| Thrombus Burden | ↓ >75% vs. | ↓ >75% vs. | ↓ >75% vs. | ↓ >75% vs. |
| | control | control | control | control |
| RVSP | >75% ↓ after ADP | <25 mmHg | <25 mmHg | <40 mmHg |
| | vs. control | | | |
| PVR (WU) | <3.0 | <3.0 | <3.0 | <3.0 |
| CO (mL/min) | >120 | >120 | >120 | >120 |
| TAPSE (mm) | — | >1.0 | >3.5 | >3.5 |
| % obstruction of | — | — | <20% | — |
| PA by CT | | | | |
| 2° End-points | | | | |
| Hgb | >14 mg/dL | >15 gm/dL | >15 gm/dL | >15 gm/dL |
| Platelet count | >9 × 10⁵/μL | >900 × 10³/μL | >900 × 10³/μL | >900 × 10³/μL |

| | |
|---|---|
| PT | <10% of control |
| PTT | <10% of control |
| Xa level | <10% of control |

Summary study end-points presented by criterion for success in each PVTE model.
PE, pulmonary embolism;
CTEPH, chronic thromboembolic pulmonary arterial hypertension;
RVSP, right ventricular systolic hypertension;
PAH, pulmonary vascular resistance;
WU, Wood units;
CO, cardiac output; pressure;
PVR, pulmonary thromboplastin time;
PTT, partial thromboplastin time;
PA, Hgb, hemoglobin;
PT, pulmonary artery

PVTE Prevention. In a disease prevention protocol, mAb-N9s or vAb control will be administered by tail vein injection at a dose on the following schedule for each model: (A) 10 min prior to ADP infusion, (B) on day 0, 3, 6, and 9 of the 10 day total protocol, (C) day 0, 7, 14, 21, and 25 of the 28-day total protocol, and (D) day 0, 7, 12, and 18 of the 21-day total protocol. See FIG. 14.

PVTE Reversal. In a disease reversal protocol, mAb-N9s or vAb control will be administered by tail vein injection at a dose on the following schedule, after the onset of thrombotic injury and vascular remodeling in each model, respectively: (A) 10 min after ADP infusion, (B) on day 6 and 8 of the 10-day total protocol, (C) day 14, 18, 21, and 25 of the 28-day total protocol, and (D) day 10, 15, and 18 of the 21-day total protocol. See FIG. 14.

### REFERENCES

1. Goldhaber S Z, Piazza G. Chronic thromboembolic pulmonary hypertension. N Engl J Med. 2011; 364:351-60.
2. Mayer E, Jenkins D, Lindner J, D'Armini A, Kloek J, Meyns B, Ilkjaer L B, Klepetko W, Delcroix M, Lang I., Pepke-Zaba J, Simonneau G, Dartevelle P. Surgical management and outcome of patients with chronic thromboembolic pulmonary hypertension: Results from an international prospective registry. J Thorac Cardiovasc Surg. 2011; 141:702-710.
3. Freed D H, Thomson B M, Berman M, Tsui S S L, Dunning J, Sheares K K, Pepke-Zaba J, Jenkins, D P. Survival after pulmonary thromboendarterectomy: Effect of residual pulmonary hypertension. J Thorac Cardiovasc Surg. 2011; 141, 383-387.
4. Brilakis E S, Manginas A N, Cokkinos D V. Chronic thromboembolic pulmonary hypertension in a patients with heterozygous for both factor V and G20210 prothombin mutation. Heart. 2001; 86:149.
5. Lang I M, Dorfmuller P, Noordegraaf A V. The Pathobiology of Chronic Thromboembolic Pulmonary Hypertension. Ann Am Thorac Soc. 2016; 13, S215-S221.
6. Colorio C C, Martinuzzo M E, Forastiero R R, Pombo G, Adamczuk Y, Carreras L O. Thrombophilic factors in chronic thromboembolic pulmonary hypertension. Blood Coagul Fibrinolysis. 2001; 12:427-32.
7. Maruoka M, Sakao S, Kantake M, Tanabe N, Kasahara Y, Kurosu K, Takiguchi Y, Masuda M, Yoshino I, Voelkel N F, Tatsumi. Characterization of myofibroblasts in chronic thromboembolic pulmonary hypertension. Int J Cardiol. 2012; 159:119-27.
8. Kim S-H, Xia D, Kim S-W, Holla V, Menter D G, Dubois R N. Human enhancer of filamentation 1 Is a mediator of hypoxia-inducible factor-lalpha-mediated migration in colorectal carcinoma cells. Cancer Res. 2010; 70, 4054-4063.
9. Giampieri S, Manning C, Hooper S, Jones L, Hill C S, Sahai E. Localised and reversible TGFβ signaling switches breast cancer from cohesive to single cell motility. Nat Cell Biol. 2009; 11:1287-96.
10. Bochenek M, Rosinus N, Lankeit M, Hobohm L, Bremmer F, Schütz E, Klok F, Horke S, Wiedenroth C, Münzel T, Lang I M, Mayer E, Konstantinides S, Schafer K. From thrombosis to fibrosis in chronic thromboembolic pulmonary hypertension. Thromb Haemost. 2017; 117:769-783.
11. Samokhin A O, Stephens B A, Wertheim B M, Wang R-S, Vargas S O, Yung L-M, Cao M, Brown M, Arons E, Dieffenbach P B, Fewell J G, Matar M, Bowman F P, Haley K J, Alba G A, Marino S M, Kumar R, Rosas I O, Waxman A B, Oldham W M, Khanna D, Graham B B, Seo S, Gladyshev V N, Yu P B, Fredenburgh L E, Loscalzo J, Leopold J A, Maron B A. NEDD9 targets COL3A1 to promote endothelial fibrosis and pulmonary arterial hypertension. Sci Transl Med. 2018; 10:445.
12. Maron B A, Oldham W M, Chan S Y, Vargas S O, Arons E, Zhang Y Y, Loscalzo J, Leopold J A. Upregulation of steroidogenic acute regulatory protein by hypoxia stimulates aldosterone synthesis in pulmonary artery endothelial cells to promote pulmonary vascular fibrosis. Circulation. 2014 Jul. 8; 130(2):168-79.
13. Italiano J E, Richardson J L, Patel-Hett S, Battinelli E, Zaslavsky A, Short S, Ryeom S, Folkman J, Klement G L. Angiogenesis is regulated by a novel mechanism: pro- and antiangiogenic proteins are organized into separate platelet alpha granules and differentially released. Blood. 2008; 111:1227-33.
14. Stevens J M. Platelet Adhesion Assays Performed Under Static Conditions. In Methods in Molecular Biology, vol. 272: Platelets and Megakaryocytes, Vol. 1: Functional Assays. Ed Gibbins J M, Mahaut-Smith M P. Humana Press Inc., Totowa, N.J.
15. Comhair S A, Xu W, Mavrakis L, Aldred M A, Asosingh K, Erzurum S C. Human primary lung endothelial cells in culture. Am J Respir Cell Mol Biol. 2012; 46: 723-730.
16. Bradbury P, Bach C T, Paul A, O'Neill G M. Src kinase determines the dynamic exchange of the docking protein NEDD9 (neural precursor cell expressed developmentally down-regulated gene 9) at focal adhesions. J Biol Chem. 2014; 289:24792-800.
17. Yen R C K, Ho T, WC, Blajchman M A. A new hemostatic agent: Thrombospheres shorten bleeding time in thrombocytopenic rabbits. Thromb Haemost. 1994; 73:986.
18. Sako D, Comess K M, Barone K M, Camphausen R T, Cumming D A, Shaw G D. A sulfated peptide segment at the amino terminus of PSGL-1 is critical for P-selectin binding. Cell. 1995; 83, 323-331.
19. Bellia F, Lanza V, Garcia-Vinuales S, Ahmed I M M, Pietropaolo A, Iacobucci C, Malgieri G, D'Abrosca G, Fattorusso R, Nicoletti V G, Sbardella D, Tundo G R, Coletta M, Pirone L, Pedone E, Calcagno D, Grasso G, Milardi D. Ubiquitin binds the amyloid R peptide and interferes with its clearance pathway. Chem Sci. 2019; 10:2732-42.
20. Jin R C, Mahoney C E, Coleman Anderson L, Ottaviano F, Croce K, Leopold J A, Zhang Y Y, Tang S S, Handy D E, Loscalzo J. Glutathione peroxidase-3 deficiency promotes platelet-dependent thrombosis in vivo. Circulation. 2011; 123:1963-73.
21. Yaoita N, Shirakawa R, Fukumoto Y, Sugimura K, Miyata S, Miura Y, Nochioka K, Miura M, Tatebe S, Aoki T, Yamamoto S, Satoh K, Kimura T, Shimokawa H, Horiuchi H. Platelets are highly activated in patients of chronic thromboembolic pulmonary hypertension. Arterioscler Thromb Vasc Biol. 2014; 34:2486-94.
22. Satoh T, Satoh K, Yaoita N, Kikuchi N, Omura J, Kurosawa R, Numano K, Al-Mamun E, Siddique M A H, Sunamura S, Nogi M, Suzuki K, Miyata S, Morser J, Shimokawa H. Activated TAFI Promotes the development of chronic thromboembolic pulmonary hypertension: A possible novel therapeutic target. Circ Res. 2017; 120: 1246-1262.
23. Aguero J, Ishikawa K, Fish K M, Hammoudi N, Hadri L, Garcia-Alvarez A, Ibanez B, Fuster V, Hajjar R J, Leopold J A. Combination proximal pulmonary artery coiling and distal embolization induces chronic elevations in pulmonary artery pressure in Swine. PLoS One. 2015; 10:e0124526.
24. Mercier O, Arthur Ataam J, Langer N B, Dorfmuller P, Lamrani L, Leceref F, Decante D, Dartevelle P, Eddahibi S, Fadel E. Abnormal pulmonary endothelial cells may underlie the enigmatic pathogenesis of chronic thromboembolic pulmonary hypertension. J Heart Lung Transpl. 2017; 36:305-14.
25. Lang I M, Marsh J J, Olman M A, Moser K M, Loskutoff D J, Schleef R R. Expression of type 1 plasminogen activator inhibitor in chronic pulmonary thrombemboli. Circulation. 1994; 89:2715-21.
26. Robbins I M, Pugh M E, Hemnes A R. Update on chronic thromboembolic pulmonary hypertension. Trends in Cardiovasc Med. 2017; 27:29-37.
27. Li J F, Lin Y, Yang Y H, Gan H L, Liang Y, Liu J, Yang S Q, Zhang W J, Cui N, Zhao L, Zhai Z G, Wang J, Wang C. Fibrinogen Aa Thr312Ala polymorphism is associated with chronic thromboembolic pulmonary hypertension. PLoS One. 2013; 8:e69635.
28. Suntharalingam J, Goldsmith K, van Marion V, Long L, Treacy C M, Dudbridge F, Toshner M R, Pepke-Zaba J, Eikenboom J C, Morrell N W. Fibrinogen Aalpha Thr312Ala polymorphism is associated with chronic thromboembolic pulmonary hypertension. Eur. Respir. J 2008; 31:736-41.
29. Bonderman D, Turecek P L, Jakowitsch J, Weltermann A, Adlbrecht C, Schneider B, Kneussl M, Rubin L J, Kyrle P A, Klepetko W, Maurer G, Lang I M. High prevalence of elevated clotting factor VIII in chronic thromboembolic pulmonary hypertension. Thromb Haemost. 2003; 90:372-376.
30. Kim M, Gans J D, Nogueira C, Wang A, Paik J-H, Feng B, Brennan C, Hahn W C, Cordon-Cardo C, Wagner S N, FLotte T J, Duncan L M, Granter S R, Chin L. Comparative oncogenomics identifies NEDD9 as a melanoma metastasis gene. Cell. 125:2006:1269-81.
31. Chung T, Connor D, Joseph J, Emmett L, Mansberg R, Peters M, Ma D, Kritharides L. Platelet activation in acute pulmonary embolism. J Thromb Haeomst. 2007; 5:918-24.
32. Novoyatleva T, Kojonazarov B, Owczarek A, Veeroju S, Rai N, Henneke I, Bohm M, Grimminger F, Ghofrani H A, Seeger W, Weissman N, Schermuly R T. Evidence for flucoidan-P-selectin axis as a therapeutic target on hypoxia-induced pulmonary hypertension. Am J Respir Crit Care Med. 2018 Epub ahead of print.
33. Federici A B, Bader R, Pagani S, Colibretti M L, De Marco L, Mannucci P M. Binding of von Willebrand factor to glycoproteins Ib and IIb/IIIa complex: affinity is related to multimeric size. Br J Haematol. 1989; 73:93-9.
34. Tikhmyanova N, Little J L, Golemis E A. Cas proteins in normal and pathological cell growth control. Cell Mol Life Sci. 2010; 67:1025-48.
35. Jujo T, Tanabe N, Sakao S, Ishibashi-Ueda H, Ishida K, Naito A, Kato F, Takeuchi T, Sekine A, Nishimura R, Sugiura T, Shigeta A, Masuda M, Tatsumi K. Severe pulmonary arteriopathy is associated with persistent hypoxemia after pulmonary endarterectomy in chronic thromboembolic pulmonary hypertension. PLoS One. 2016; 11, e0161827.
36. Pugacheva E N, Golemis E A. The focal adhesion scaffolding protein HEF1 regulates activation of the Aurora-A and Nek2 kinases at the centrosome. Nat Cell Biol. 2005; 7:937-946.
37. Law S F, Zhang Y Z, Klein-Szanto A J P, Golemis E A. Cell cycle-regulated processing of HEF1 to multiple protein forms differentially targeted to multiple subcellular compartments. Mol Cell Biol. 1998; 18, 3540-3551.
38. Maron B A, Zhang Y Y, Handy D E, Beuve A, Tang S S, Loscalzo J, Leopold J A. Aldosterone increases oxidant stress to impair guanylyl cyclase activity by cysteinyl thiol oxidation in vascular smooth muscle cells. J Biol Chem. 2009; 284:7665-72.
39. Samokhin A O, Stephens B A, Wertheim B M, Wang R-S, Vargas S O, Yung L-M, Cao M, Brown M, Arons E, Dieffenbach P B, Fewell J G, Matar M, Bowman F P, Haley K J, Alba G A, Marino S M, Kumar R, Rosas I O, Waxman A B, Oldham W M, Khanna D, Graham B B, Seo S, Gladyshev V N, Yu P B, Fredenburgh L E, Loscalzo J, Leopold J A, Maron B A. NEDD9 targets COL3A1 to promote endothelial fibrosis and pulmonary arterial hypertension. Sci Transl Med. 2018; 10:445.
40. Schindelin J, Arganda-Carreras I, Frise E, Kaynig V, Longair M, Pietzsch T, Preibisch S, Rueden C, Saalfeld S, Schmid B, Tinevez J Y, White D J, Hartenstein V, Eliceiri K, Tomancak P, Cardona A. Fiji: an open-source platform for biological-image analysis. Nat Methods. 2012; 9(7): 676-682. PMID 22743772.
41. Italiano J E Jr, Richardson J L, Patel-Hett S, Battinelli E, Zaslavsky A, Short S, Ryeom S, Folkman J, Klement G L., Angiogenesis is regulated by a novel mechanism: pro- and antiangiogenic proteins are organized into separate platelet alpha granules and differentially released. Blood 2008; 111(3):1227-1233.
42. Peng, J, Gygi S P. Proteomics: The move to mixtures. J Mass Spec. 2001; 36:1083-91.
43. Eng J K, McCormack A L, Yates J R. An approach to correlate tandem mass spectral data of peptides with amino acid sequences in a protein database. J Am Soc Mass Spectrom. 1994; 5:976-989.
44. Maron B A, Oldham W M, Chan S Y, Vargas S O, Arons E, Zhang Y Y, Loscalzo J, Leopold J A. Upregulation of steroidogenic acute regulatory protein by hypoxia stimulates aldosterone synthesis in pulmonary artery endothelial cells to promote pulmonary vascular fibrosis. Circulation. 2014 Jul. 8; 130(2):168-79.
45. Seo S, Asai T, Saito T, Suzuki T, Morishita Y, Nakamoto T, Ichikawa M, Yamamoto G, Kawazu M, Yamagata T, Sakai R, Mitani K, Ogawa S, Kurokawa M, Chiba S, Hirai H. Crk-associates substrate lymphocyte type is required for lymphocyte trafficking and marginal zone B cell maintenance. J Immunol. 2005; 175:3492-501.
46. Jin R C, Mahoney C E, Coleman Anderson L, Ottaviano F, Croce K, Leopold J A, Zhang Y Y, Tang S S, Handy D E, Loscalzo J. Glutathione peroxidase-3 deficiency promotes platelet-dependent thrombosis in vivo. Circulation. 2011; 123:1963-73.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. An antibody that binds specifically to human neural precursor cell expressed, developmentally down-regulated 9 (NEDD9) at an epitope in or near a NEDD9 substrate domain, preferably a tyrosine rich substrate domain that is accessible on the extracellular HPAEC plasma membrane, preferably a substrate domain that comprises one or more YxxP motifs.

2. The antibody of claim 1, which binds NEDD9 within one of the following sequences: NEDD9 AA 75-125: EQPASG LMQQTFGQQK LYQVPNPQAA PRDTIYQVPP SYQNQGIYQV PTGHG (SEQ ID NO:1); or NEDD9 AA 175-225: DVYDIP PSHTTQGVYD IPPSSAKGPV FSVPVGEIKP QGVYDIPPTK GVYAI (SEQ ID NO:2).

3. The antibody of claim 2, which binds NEDD9 in substrate domain P1,

4. The antibody of claim 3, which binds NEDD9 within the sequence LYQVPNPQAAPR (SEQ ID NO:3).

5. The antibody of claim 2, which binds NEDD9 within substrate domain P2.

6. The antibody of claim 5, which binds NEDD9 within the sequence GPVFSVPVGEIKPQGVYDIPPTK (SEQ ID NO:4).

7. An antibody that binds specifically to NEDD9, obtained from a mammal that has been immunized with a peptide comprising NEDD9 substrate domain P1 (LYQVPNPQAAPR) (SEQ ID NO:3) or NEDD9 substrate domain P2 (GPVFSVPVGEIKPQGVYDIPPTK; SEQ ID NO:4).

8. The antibody of claim 1, which is a monospecific polyclonal antibody or a monoclonal antibody.

9. The antibody of claim 1, which reduces or blocks formation of binding complexes between NEDD9 and p-Selectin; reduces binding affinity of a protein-protein complex between NEDD9 and P-Selectin; and/or reduce PVTE formation and/or platelet-endothelial adhesion.

10. A composition comprising an antigen-binding portion of the antibody of claim 1.

11. A method of generating an antibody that binds to an epitope in NEDD9 substrate domain, the method comprising immunizing a mammal with a peptide comprising a sequence that is at least 80% identical to at least 10 consecutive amino acids from:
(i) the NEDD9 substrate domain P1, preferably a peptide comprising LYQVPNPQAAPR (SEQ ID NO:3), LYQVPNPQAAPRDT-amide (SEQ ID NO:5), or CFGQQKLYQVPNPQAAPRDT-amide (SEQ ID NO:6), or
(ii) NEDD9 substrate domain P2, preferably a peptide comprising GEIKPQGVYDIPPTKGV (SEQ ID NO:7) or CGEIKPQGVYDIPPTKGV-amide (SEQ ID NO:8), optionally wherein the peptide is modified to increase antigenicity, and collecting antibodies from the mammal.

12. The method of claim 11, wherein the peptide is modified to increase antigenicity, preferably wherein the peptide is conjugated to one or both of keyhole limpet hemocyanin or ovalbumin.

13. The method of claim 12, further comprising:
isolating the blood serum from the immunized mammal containing antibodies;
isolating antibody-producing cells taken from the spleen or lymph node of the immunized mammal;
fusing the isolated antibody-producing cells with myeloma cells resulting in a hybridoma;
cloning the hybridoma and recovering antibody from the culture thereof to yield a monoclonal antibody; and
purifying the monoclonal antibodies using NEDD9 or a peptide therefrom.

14. An antibody that binds specifically to NEDD9, generated by the method of claim 1.

15. The antibody of claim 14, which reduces or blocks formation of binding complexes between NEDD9 and p-Selectin; reduces binding affinity of a protein-protein complex between NEDD9 and P-Selectin; and/or reduce PVTE formation and/or platelet-endothelial adhesion.

16. A method of reducing platelet-endothelial adhesion in a subject in need thereof, the method comprising administering to the subject a therapeutically effective amount of the antibody of claim 1.

17. A method of treating, or reducing risk of, pulmonary vascular thromboembolism (PVTE) in a subject in need thereof, the method comprising administering to the subject a therapeutically effective amount of the antibody of claim 1.

18. The method of claim 16, wherein the subject has, or is at risk of developing, luminal pulmonary embolism (PE), cancer-associated PE, pulmonary arterial hypertension (PAH), or chronic thromboembolic pulmonary hypertension (CTEPH).

19. The method of claim 16, further comprising treating the subject with one or more of anticoagulation (optionally using warfarin, direct oral anticoagulants), systemic thrombolysis, catheter-directed thrombolysis, or surgical clot resection.

20. The method of claim 16, wherein the antibody is administered parenterally or orally.

21. (canceled)

22. (canceled)

23. (canceled)

24. (canceled)

25. (canceled)

26. A pharmaceutical composition comprising the antibody of claim 1, and a carrier.
